(19) United States
(12) Patent Application Publication
(10) Pub. No.: US 2014/0161228 A1
(43) Pub. Date: Jun. 12, 2014

(54) RADIOGRAPHY SYSTEM AND RADIOGRAPHY METHOD

(71) Applicant: FUJIFILM Corporation, Tokyo (JP)

(72) Inventors: Kouichi KITANO, Ashigarakami-gun (JP); Naoyuki NISHINO, Ashigarakami-gun (JP); Yasunori OHTA, Ashigarakami-gun (JP); Naoto IWAKIRI, Ashigarakami-gun (JP); Haruyasu NAKATSUGAWA, Ashigarakami-gun (JP)

(73) Assignee: FUJIFILM Corporation, Tokyo (JP)

(21) Appl. No.: 14/179,219

(22) Filed: Feb. 12, 2014

Related U.S. Application Data
(63) Continuation of application No. PCT/JP2012/071384, filed on Aug. 24, 2012.

(30) Foreign Application Priority Data
Aug. 25, 2011 (JP) 2011-183513

(51) Int. Cl. A61B 6/00

(52) U.S. Cl. CPC A61B 6/542
USPC 378/62

(57) ABSTRACT

In a radiography system and radiography method according to the present invention, the radiography system comprises: a radiography device further comprising a radiation device further comprising a radiation source, and a radiation detection device which converts radiation which passes through a radiography subject into radiography information; and a system control portion which controls the radiography device to execute radiography at a set frame rate. The system control portion further comprises: a radiation emission disabling portion which interrupts the irradiation of radiation from the radiation source at least in a case where an error occurs with the radiography device; and a recovery processing portion which implements control so as to set the irradiation energy of the radiation source to a preset low irradiation energy and execute the radiography in a case where recovering from the error state.

### CROSS-REFERENCE TO RELATED APPLICATIONS AND PRIORITY CLAIM

This application is a Continuation of International Application No. PCT/JP2012/071384 filed on Aug. 24, 2012, which was published under PCT Article 21(2) in Japanese, which is based upon and claims the benefit of priority from Japanese Patent Application No. 2011-183513 filed on Aug. 25, 2011, the contents all of which are incorporated herein by reference.

### TECHNICAL FIELD

The present invention relates to a radiographic image capturing system (radiography system) and a radiographic image capturing method (radiography method) for obtaining a radiographic moving image by performing a radiographic image capturing process at a specified frame rate using a radiographic image capturing apparatus.

### BACKGROUND ART

Recently, it has become necessary in surgery, contrast-enhanced radiography, and in treatments for bone fractures, etc., to read radiographic image information of a patient from a radiation detector, and to display the radiographic image information immediately after the radiographic image information has been captured for the purpose of quickly and adequately treating the patient. One radiation detector, which has been developed to meet such a demand, is known as a flat panel detector (FPD) having solid-state detecting elements (hereinafter referred to as “pixels”) for converting radiation directly into electric signals, or alternatively, for converting radiation into visible light with a scintillator and then converting the visible light into electric signals to read radiographic image information represented by the radiation.

There has been proposed an X-ray image diagnosing apparatus, which displays a radiographic moving image on a monitor by performing a radiographic image capturing process at a specified frame rate, so that an observer can grasp in real time how a catheter, for example, enters into a subject (see, for example, Japanese Laid-Open Patent Publication No. 2005-087633).

Heretofore, there also has been proposed an X-ray image diagnosing apparatus, which makes it unnecessary to perform an image capturing process again on a patient, and hence prevents the patient from being exposed to excessive X-rays, even in a case where an image processing circuit and an image data storage device suffer from an error while a captured image is being displayed in real time (see Japanese Laid-Open Patent Publication No. 2008-284090). Further, an X-ray image diagnosing apparatus is known, which controls a subsequent X-ray image capturing process depending on the purpose thereof, even in the event of a transmission failure of operational instruction information from a control portion (see Japanese Laid-Open Patent Publication No. 2009-297304).

### SUMMARY OF INVENTION

According to Japanese Laid-Open Patent Publication No. 2008-284090 and Japanese Laid-Open Patent Publication No. 2009-297304, radiographic image information is secured upon the occurrence of an error in an X-ray image diagnosing apparatus, and a radiographic image capturing process is continued in a preset operation mode in the event that transmission of operational instruction information from a control portion is interrupted. However, these publications are silent concerning what type of processing sequence should be carried out for recovering from an error, and take nothing whatsoever into account concerning performance of a recovery process while reducing the risk of suffering from a reoccurring error and minimizing the burden on a subject, e.g., a patient.

The present invention has been made in view of the aforementioned difficulties. It is an object of the present invention to provide a radiographic image capturing system and a radiographic image capturing method for performing a process to handle errors, and to perform a recovery process while reducing the risk of suffering from a reoccurring error and minimizing the burden on a subject, e.g., a patient, due to recovery from the error.

[1] A radiographic image capturing system according to a first aspect of the invention comprises a radiographic image capturing apparatus having a radiation device including a radiation source and a radiation detecting device for converting radiation, which is emitted from the radiation source and transmitted through a subject, into radiographic image information, and a system control portion for controlling the radiographic image capturing apparatus to carry out a radiographic image capturing process at a set frame rate, wherein the system control portion includes a radiation emission disabling portion for stopping the radiation source from emitting radiation in a case where an error has occurred in at least the radiographic image capturing apparatus, and a recovery processing portion for carrying out a radiographic image capturing process while setting an irradiation energy level of the radiation source to a preset low irradiation energy level upon recovery of the radiographic image capturing apparatus from the error.

According to the present invention, in a case where an error has occurred in at least the radiographic image capturing apparatus, the radiation source is stopped from emitting radiation. In a case where the radiographic image capturing apparatus recovers from the error, the radiographic image capturing apparatus continues to capture radiographic images (a radiographic moving image) at the set frame rate. This differs significantly from the technology disclosed in Japanese Laid-Open Patent publication No. 2009-297304, i.e., a technology in which, in a case where a control signal fails to be transmitted from the console, exposure to radiation is continued in a predetermined way. This is because the technology disclosed in Japanese Laid-Open Patent Publication No. 2009-297304 does not assume that an error has occurred in the control system for the radiation source.

According to the present invention, in a case where the radiographic image capturing apparatus recovers from the error, the recovery processing portion sets the irradiation energy level of the radiation source to the preset low irradiation energy level, and thereafter, the radiographic image capturing process is carried out. Even in a case where the radiographic image capturing apparatus is judged as having recovered from an error, the radiographic image capturing apparatus actually may not have fully recovered from the error, i.e., the error may still remain unremoved. In a case where the irradiation energy level of the radiation source is set to an ordinary energy level or a high energy level prior to the occurrence of the error during a time that the radiographic image capturing apparatus has not yet fully recovered from the error, then the radiographic image capturing apparatus runs the risk of suffering from a reoccurring error. According to the present invention, as described above, since the irradiation energy level of the radiation source is set to the preset low energy level, the risk of suffering from a reoccurring error is reduced, and the radiographic image capturing system can quickly be brought back to a state for capturing a radiographic moving image. In addition, the burden posed on the subject due to undue exposure to radiation is reduced.

[2] In the first aspect of the present invention, the recovery processing portion may set a radiation dose per irradiation event from the radiation source to a level lower than a radiation dose per irradiation event immediately prior to occurrence of the error.

[3] In the first aspect of the present invention, the recovery processing portion may set a number of irradiation events per unit time performed by the radiation source to a value lower than a number of irradiation events per unit time prior to occurrence of the error.

[4] In the first aspect of the present invention, the recovery processing portion may set the total irradiation energy level per unit time of the radiation source to a low level.

[5] In the first aspect of the present invention, the recovery processing portion may set a radiation dose per irradiation event from the radiation source to a level lower than a radiation dose per irradiation event prior to the occurrence of the error, and may set a number of irradiation events per unit time performed by the radiation source to a value lower than a number of irradiation events per unit time prior to the occurrence of the error.

[6] In the first aspect of the present invention, the recovery processing portion may set the irradiation energy level of the radiation source to a lowest irradiation energy level from among a plurality of irradiation energy levels set within a predetermined period in past.

[7] In the first aspect of the present invention, the radiographic image capturing apparatus may further include a radiation source control portion for controlling the radiation source based on a command from the system control portion, wherein the radiation emission disabling portion may supply a disable signal for disabling emission of radiation to the radiation source control portion, and the radiation source control portion may stop the radiation source from emitting radiation based on the disable signal supplied from the radiation emission disabling portion.

[8] In [7], the radiographic image capturing apparatus may further include a detecting device control portion for controlling the radiation detecting device based on a command from the system control portion, wherein the system control portion may send an error notification to the detecting device control portion after the disable signal has been supplied from the radiation emission disabling portion, and the detecting device control portion may stop controlling at least the radiation detecting device based on the error notification sent from the system control portion.

[9] In the first aspect of the present invention, the radiographic image capturing apparatus may further include a radiation source control portion for controlling the radiation source based on a command from the system control portion, wherein the radiation emission disabling portion may stop supply of an exposure start signal for emitting radiation to the radiation source control portion.

[10] In [9], the radiographic image capturing apparatus may further include a detecting device control portion for controlling the radiation detecting device based on a command from the system control portion, wherein the system control portion may send an error notification to the detecting device control portion after the radiation emission disabling portion has stopped supply of the exposure start signal, and the detecting device control portion may stop controlling the radiation detecting device based on the error notification sent from the system control portion.

[11] In [7] through [10], based on the recovery from the error, the recovery processing portion may supply information concerning setting of the irradiation energy level of the radiation source to the low irradiation energy level to the radiation device, and may supply parameter information concerning the recovery from the error to the detecting device control portion, and the system control portion may resume operation of the radiation device and the radiation detecting device.

[12] In the first aspect of the present invention, the radiographic image capturing system may further comprise a display device for displaying radiographic image information captured by the radiographic image capturing process that is carried out at the set frame rate, wherein in the case where the error has occurred, the system control portion controls the display device to display radiographic image information captured immediately prior to occurrence of the error at the set frame rate, during a period from the occurrence of the error to the recovery from the error.

[13] According to a second aspect of the invention, there also is provided a radiographic image capturing method for carrying out a radiographic image capturing process at a set frame rate with a radiographic image capturing apparatus including a radiation source and a radiation detecting device for converting radiation, which is emitted from the radiation source and transmitted through a subject, into radiographic image information, comprising the steps of stopping the radiation source from emitting radiation in a case where an error has occurred in at least the radiographic image capturing apparatus, and carrying out a radiographic image capturing process while setting an irradiation energy level of the radiation source to a preset low irradiation energy level upon recovery of the radiographic image capturing apparatus from the error.

With the radiographic image capturing system and the radiographic image capturing method according to the present invention, as described above, in addition to the process performed upon occurrence of an error, a recovery process is performed to recover the radiographic image capturing apparatus from the error, while at the same time reducing the risk of reoccurring errors as well as reducing the burden on the subject, e.g., a patient.

### BRIEF DESCRIPTION OF DRAWINGS

FIG. 1 is a schematic view of a radiographic image capturing system according to a first embodiment of the present invention (first radiographic image capturing system);

FIG. 2 is a block diagram of a radiation device and a radiation detecting device of the first radiographic image capturing system;

FIG. 3 is a circuit diagram showing a configuration of the radiation detecting device, and in particular, a configuration of a radiation detector;

FIG. 4 is a block diagram showing a configuration primarily of a system control portion of the first radiographic image capturing system;

FIG. 5 is a flowchart (part 1) of a processing sequence of the first radiographic image capturing system;

FIG. 6 is a flowchart (part 2) of the processing sequence of the first radiographic image capturing system;

FIG. 7 is a timing chart of the processing sequence of the first radiographic image capturing system;

FIG. 8 is a block diagram showing a configuration primarily of a system control portion of a radiographic image capturing system according to a second embodiment of the present invention (second radiographic image capturing system);

FIG. 9 is a flowchart of selected steps of a processing sequence of the second first radiographic image capturing system;

FIG. 10 is a timing chart of a processing sequence of the second radiographic image capturing system;

FIG. 11 is a block diagram showing a configuration primarily of a system control portion of a radiographic image capturing system according to a third embodiment of the present invention (third radiographic image capturing system);

FIG. 12 is a block diagram showing a configuration primarily of a system control portion of a radiographic image capturing system according to a fourth embodiment of the present invention (fourth radiographic image capturing system);

FIG. 13 is a timing chart of a processing sequence of the fourth radiographic image capturing system;

FIG. 14 is a cross-sectional view of a configuration made up of three pixels of a radiation detector according to a modification of the present invention; and

FIG. 15 is a cross-sectional view of a thin-film transistor (TFT) and an electric charge accumulator shown in FIG. 14.

### DESCRIPTION OF EMBODIMENTS

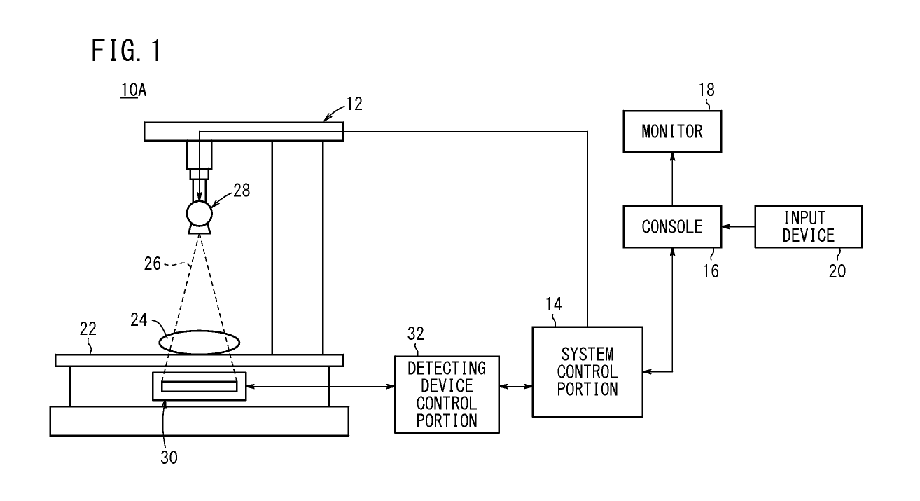

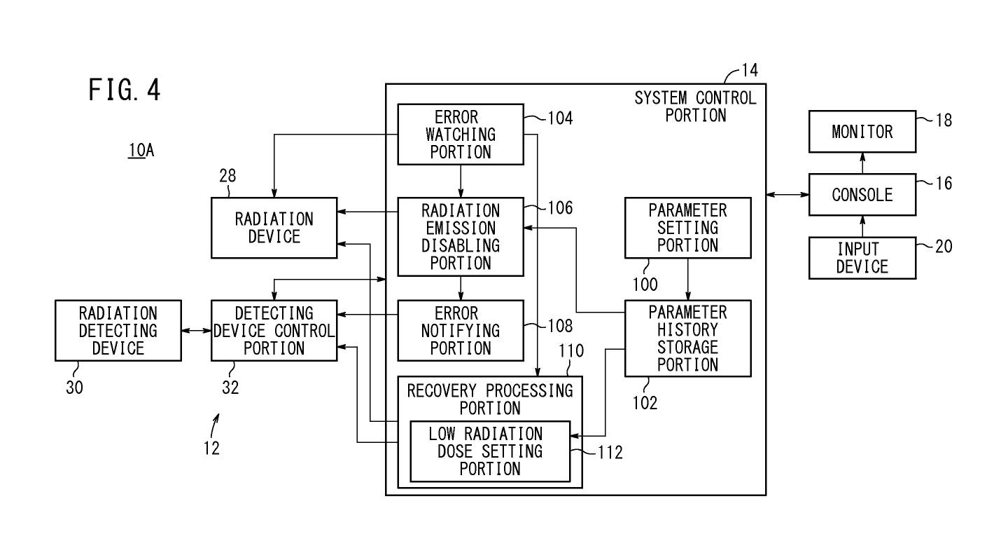

Radiographic image capturing systems and radiographic image capturing methods according to preferred embodiments of the present invention will be described below with reference to FIGS. 1 through 15.

As shown in FIG. 1, a radiographic image capturing system according to a first embodiment of the present invention (hereinafter referred to as a “first radiographic image capturing system 10A”) includes a radiographic image capturing apparatus 12 and a system control portion 14 for controlling the radiographic image capturing apparatus 12 to perform a radiographic image capturing process at a specific frame rate in a range from 15 frames/second to 60 frames/second, for example. The system control portion 14 is connected to a console 16 for carrying out data communications therewith. The console 16 is connected to a monitor 18 (display device) for enabling observation of images and image diagnosis, and an input device 20, e.g., a keyboard, a mouse, etc., for entering control inputs. Using the input device 20, the operator, e.g., a doctor or a radiological technician, specifies a dose of radiation to be applied and the frame rate of a radiographic image capturing process, which are suitable for the present situation, for a surgical operation and a catheter insertion process to be carried out while observing a moving image being displayed. Data that have been entered using the input device 20 and data that have been generated and edited on the console 16 are supplied to the system control portion 14. Radiographic image information, etc., from the system control portion 14 is supplied to the console 16 and displayed on the monitor 18.

The radiographic image capturing apparatus 12 includes a radiation device 28 for applying radiation 26 to a subject 24 on an image capturing base 22, a radiation detecting device 30 for converting radiation 26 that has passed through the subject 24 into radiographic image information, and a detecting device control portion 32 for sending and receiving data including radiographic image information between the radiation detecting device 30 and the system control portion 14, and for controlling, e.g., moving, the radiation detecting device 30 based on commands from the system control portion 14.

The radiation detecting device 30 may be moved in a case where it is necessary to capture a radiographic image of a relatively wide range of the subject 24, e.g., to capture a radiographic moving image of the spine of the subject 24, or to capture a radiographic moving image of a region where a catheter enters into the body of the subject 24. For capturing such a radiographic image, the system control portion 14 supplies the detecting device control portion 32 with a movement control signal based on a control input entered by the operator (the doctor or the radiological technician). In response to the movement control signal from the system control portion 14, the detecting device control portion 32 controls a moving mechanism, not shown, in order to move the radiation detecting device 30.

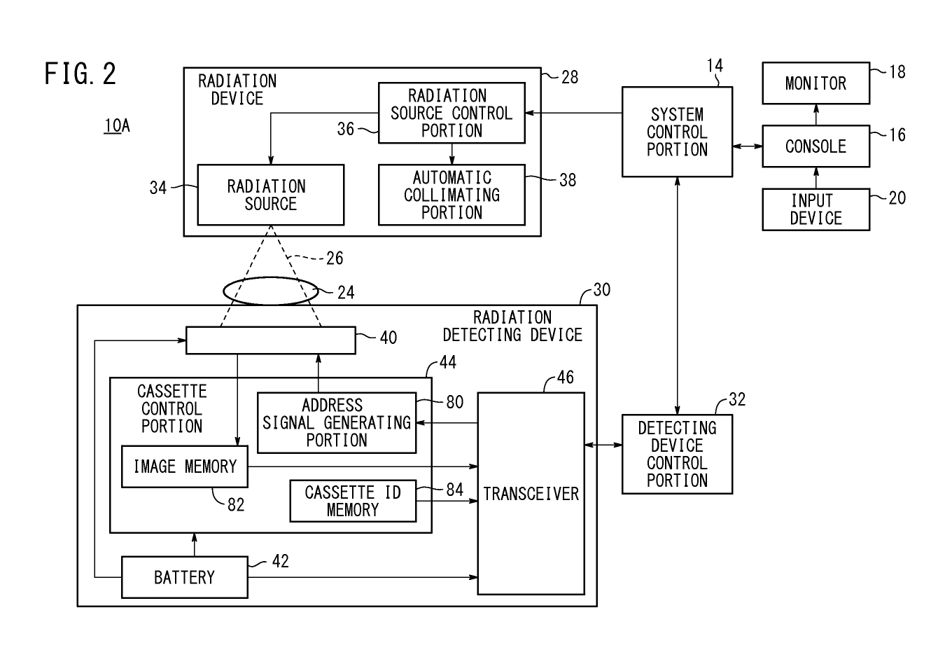

As shown in FIG. 2, the radiation device 28 has a radiation source 34, a radiation source control portion 36 for controlling the radiation source 34 based on a command from the system control portion 14, and an automatic collimating portion 38 for increasing or reducing the area to be irradiated with radiation 26 based on a command from the system control portion 14.

The radiation detecting device 30 has a radiation detector 40, a battery 42 serving as a power supply, a cassette control portion 44 for energizing the radiation detector 40, and a transceiver 46 for sending and receiving signals including radiographic image information from the radiation detector 40 to and from an external device. The radiographic image information sent from the transceiver 46 is supplied through the detecting device control portion 32 to the system control portion 14 and the console 16, and the radiographic image information is displayed on the monitor 18. In a case where a radiographic image capturing process is carried out at a specified frame rate, the system control portion 14 is supplied with successive items of radiographic image information from the detecting device control portion 32, and the system control portion 14 controls the monitor 18 to display a radiographic moving image in real time.

In order to prevent the cassette control portion 44 and the transceiver 46 from becoming damaged due to radiation 26, a lead plate or the like preferably is provided on irradiated surfaces of the cassette control portion 44 and the transceiver 46.

The radiation detector 40 may comprise an indirect-conversion-type radiation detector (a face-side readout type or a reverse-side readout type of radiation detector) for converting radiation 26 that has passed through the subject 24 into visible light with a scintillator, and then converting the visible light into electric signals with solid-state detecting elements (hereinafter referred to as “pixels”) made of a material such as amorphous silicon (a-Si) or the like. A radiation detector, which is of an ISS (Irradiation Side Sampling) type as a face-side readout type, includes solid-state detecting elements and a scintillator, which are arranged successively along a direction in which radiation 26 is applied. A radiation detector, which is of a PSS (Penetration Side Sampling) type as a reverse-side readout type, includes a scintillator and solid-state detecting elements, which are arranged successively along a direction in which radiation 26 is applied. The radiation detector 40 may alternatively comprise, rather than an indirect-conversion-type radiation detector, a direct-conversion-type radiation detector for converting a dose of radiation 26 directly into electric signals using solid-state detecting elements made of a material such as amorphous selenium (a-Se) or the like.

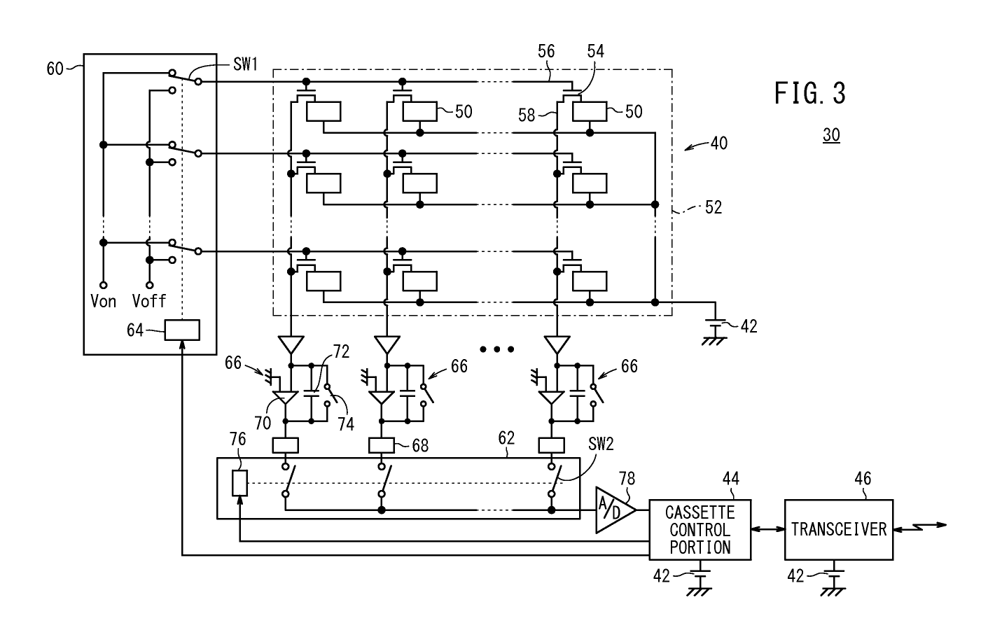

A circuit arrangement of the radiation detecting device 30, which includes an indirect-conversion-type radiation detector 40, for example, will be described in detail below with reference to FIG. 3.

The radiation detector 40 comprises an array of thin-film transistors (hereinafter referred to as “TFTs 54”) arranged in rows and columns, and a photoelectric transducer layer 52 including pixels 50 and made of a material such as a-Si or the like for converting visible light into electric signals. The photoelectric transducer layer 52 is disposed on the array of TFTs 54. The pixels 50 store electric charges, which are generated in a case where the pixels 50 convert visible light into electric signals (analog signals). The TFTs 54 are turned on successively along each row at a time, whereby the stored electric charges are read from the pixels 50 as image signals.

The TFTs 54 are connected respectively to the pixels 50. Gate lines 56, which extend in parallel with the rows, and signal lines 58, which extend in parallel with the columns, are connected to the TFTs 54. The gate lines 56 are connected to a line scanning drive portion 60, and the signal lines 58 are connected to a multiplexer 62. The gate lines 56 are supplied with control signals Von, Voff from the line scanning drive portion 60 for turning on and off the TFTs 54 along the rows. The line scanning drive portion 60 includes a plurality of switches SW1 for switching between the gate lines 56, and a first address decoder 64 for supplying a selection signal for selecting one of the switches SW1 at a time. The first address decoder 64 is supplied with an address signal from the cassette control portion 44.

The signal lines 58 are supplied with electric charges stored in the pixels 50 through the TFTs 54, which are arranged in columns. The electric charges supplied to the signal lines 58 are amplified by charge amplifiers 66. The charge amplifiers 66 are connected through respective sample and hold circuits 68 to the multiplexer 62.

The electric charges read from the columns are supplied respectively through the signal lines 58 to the charge amplifiers 66 in the columns. Each of the charge amplifiers 66 comprises an operational amplifier 70, a capacitor 72, and a switch 74. In a case where the switch 74 is turned off, the charge amplifier 66 converts a charge signal supplied to an input terminal of the operational amplifier 70 into a voltage signal, and supplies the voltage signal to the sample and hold circuit 68. The charge amplifier 66 amplifies the electric signal by a predetermined gain set in the cassette control portion 44 and supplies an amplified electric signal. Information concerning the gain of the charge amplifier 66, i.e., gain setting information, is supplied from the system control portion 14 through the detecting device control portion 32 to the cassette control portion 44. Based on the supplied gain setting information, the cassette control portion 44 sets the gain of the charge amplifier 66.

The operational amplifier 70 has another input terminal connected to GND (ground potential). In a case where the switch 74 is turned on, the electric charge stored in the capacitor 72 is discharged by a closed circuit of the capacitor 72 and the switch 74, and the electric charges stored in the pixels 50 are drained to GND (ground potential) through the closed switch 74 and the operational amplifier 70. The process of turning on the switch 74 of the charge amplifier 66 in order to discharge the electric charge stored in the capacitor 72 and to drain the electric charges stored in the pixels 50 to GND (ground potential) is referred to as a resetting process (blank reading). In the resetting process, voltage signals, which are representative of the electric charges stored in the pixels 50, are not supplied to the multiplexer 62, but rather are drained from the pixels 50.

The multiplexer 62 includes a plurality of switches SW2 for switching successively between the signal lines 58 and a second address decoder 76, for thereby outputting a selection signal for selecting one of the switches SW2 at a time. The second address decoder 76 is supplied with an address signal from the cassette control portion 44. The multiplexer 62 has an output terminal connected to an A/D converter 78. The A/D converter 78 converts radiographic image information into digital image signals, which are supplied to the cassette control portion 44.

The TFTs 54, which operate as switching devices, may be combined with another image capturing device such as a CMOS (Complementary Metal-Oxide Semiconductor) image sensor or the like. Alternatively, the TFTs 54 may be replaced with a CCD (Charge-Coupled Device) image sensor for shifting and transferring electric charges with shift pulses that correspond to gate signals in the TFTs.

As shown in FIG. 2, the cassette control portion 44 of the radiation detecting device 30 includes an address signal generating portion 80, an image memory 82, and a cassette ID memory 84.

Based on readout control information from the system control portion 14, for example, the address signal generating portion 80 supplies address signals to the first address decoder 64 of the line scanning drive portion 60, and to the second address decoder 76 of the multiplexer 62 shown in FIG. 3. The readout control information includes information representing a progressive mode, an interlace mode (an odd-numbered row readout mode, an even-numbered row readout mode, an every third row readout mode, an every fourth row readout mode, etc.), and a binning mode (a 4-pixels-into-1 readout mode, a 6-pixels-into-1 readout mode, a 9-pixels-into-1 readout mode, etc.). In a 4-pixels-into-1 readout mode, for example, two adjacent gate lines are energized simultaneously, i.e., supplied with the control signal Von, and two adjacent signal lines are energized simultaneously, thereby mixing the electric charges, which are contained in four adjacent pixels in two rows and two columns, into a single superpixel electric charge to be read. The address signal generating portion 80 generates address signals depending on the mode represented by the readout control information, and supplies the generated address signals to the first address decoder 64 of the line scanning drive portion 60 and to the second address decoder 76 of the multiplexer 62. The readout control information is generated by the system control portion 14 based on a control input entered by the operator, for example, and the readout control information is supplied to the cassette control portion 44 of the radiation detecting device 30.

The image memory 82 stores radiographic image information detected by the radiation detector 40. The cassette ID memory 84 stores cassette ID information for identifying the radiation detecting device 30. The transceiver 46 sends cassette ID information stored in the cassette ID memory 84 and radiographic image information stored in the image memory 82 through the detecting device control portion 32 to the system control portion 14 via a wired or wireless communication link.

In addition, the system control portion 14 of the first radiographic image capturing system 10A has a parameter setting portion 100, a parameter history storage portion 102, an error watching portion 104, a radiation emission disabling portion 106, an error notifying portion 108, and a recovery processing portion 110.

In the case that new parameters (dose of radiation to be applied, frame rate, etc.) are set by a control input made by the operator, the parameter setting portion 100 stores the new radiation dose, the frame rate, etc., which have been set, as latest parameters in the parameter history storage portion 102. In particular, in a case where the dose of radiation to be applied is newly set, the parameter setting portion 100 supplies first dose setting information Sa1, including information (tube voltage, tube current, image capturing time, etc.) concerning the newly set radiation dose, to the radiation device 28. In a case where a gain and a readout mode are newly set for the charge amplifiers 66, the parameter setting portion 100 supplies first readout control information Sb1, including information concerning the newly set gain and the newly set readout mode, to the detecting device control portion 32.

The parameter history storage portion 102 stores radiation doses and frame rates, which were applied over a predetermined period of time in the past from the present time, from among the radiation doses and frame rates that have been set thus far.

Based on detected signals from various non-illustrated sensors, the error watching portion 104 judges whether or not an error has occurred in at least the radiographic image capturing apparatus 12, as well as whether the radiographic image capturing apparatus 12 has recovered from the error.

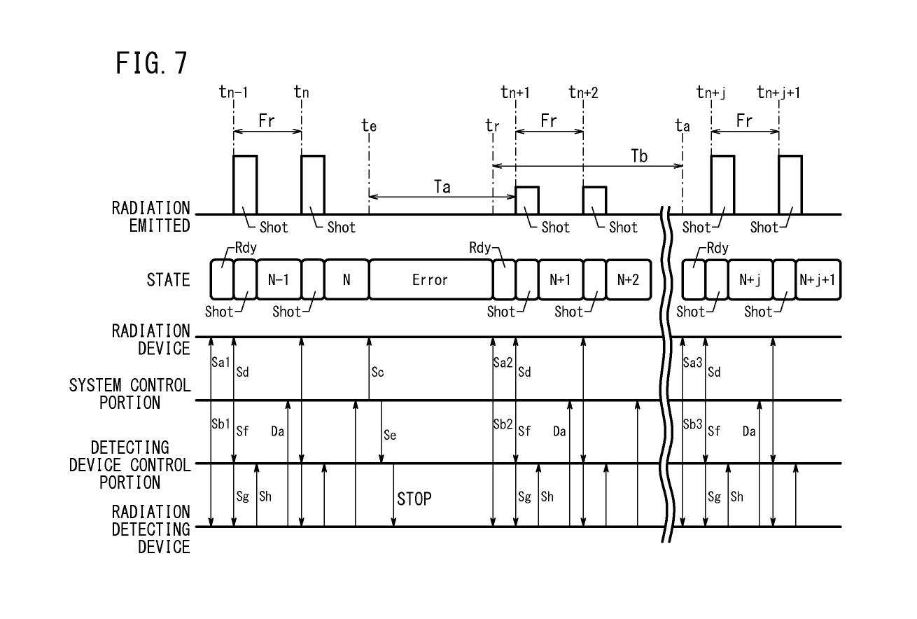

In a case where the error watching portion 104 judges that an error has occurred, then the radiation emission disabling portion 106 stops the radiation source 34 from emitting radiation. More specifically, the radiation emission disabling portion 106 supplies a disable signal Sc (see FIG. 7) for disabling emission of radiation to the radiation device 28. Alternatively, the radiation emission disabling portion 106 stops supplying an exposure start signal Sd (see FIG. 7) for initiating emission of radiation to the radiation device 28. The radiation source control portion 36 of the radiation device 28 stops the radiation source 34 from emitting radiation based on the disable signal Sc from the radiation emission disabling portion 106.

After the disable signal Sc has been supplied from the radiation emission disabling portion 106, or after the exposure start signal Sd has stopped being supplied from the radiation emission disabling portion 106, the error notifying portion 108 sends an error notification Se (see FIG. 7) to the detecting device control portion 32. In response to the error notification Se, the detecting device control portion 32 stops control of at least the radiation detecting device 30. At this time, all of the pixels may be reset.

In a case where the error watching portion 104 judges that the radiographic image capturing apparatus 12 has recovered from the error, then the recovery processing portion 110 controls the radiation device 28 to perform a radiographic image capturing process. At this time, the radiation source 34 is set to a preset low irradiation energy level.

The recovery processing portion 110 has a low radiation dose setting portion 112 for setting the dose of radiation from the radiation source 34 per irradiation event to a level lower than the dose of radiation from the radiation source 34 per irradiation event prior to the occurrence of the error. The low radiation dose setting portion 112 sets the dose of radiation from the radiation source 34 per irradiation event to a level that is in a range from ⅓ to ⅔ of the dose of radiation from the radiation source 34 per irradiation event prior to the occurrence of the error, e.g., the latest radiation dose stored in the parameter history storage portion 102. Alternatively, the low radiation dose setting portion 112 may set the radiation dose to a lower ratio, e.g., in a range from ⅕ to ⅘.

The recovery processing portion 110 supplies second dose setting information Sa2, which includes information (tube voltage, tube current, image capturing time, etc.) concerning the low radiation dose set by the low radiation dose setting portion 112, to the radiation device 28, and supplies second readout control information Sb2 (parameter information), which includes information concerning a gain and a readout mode for the charge amplifiers 66 to enable recovery, to the detecting device control portion 32.

Upon elapse of a predetermined recovery watch period (5 to 10 seconds from the time that recovery from an error is judged to have occurred), the recovery processing portion 110 supplies third dose setting information Sa3, which includes information (tube voltage, tube current, image capturing time, etc.) concerning the radiation dose (the latest radiation dose stored in the parameter history storage portion 102) immediately prior to the occurrence of the error, to the radiation device 28. The recovery processing portion 110 also supplies third readout control information Sb3, which includes information (the latest gain setting information and readout mode information stored in the parameter history storage portion 102) concerning a gain and a readout mode for the charge amplifiers 66 immediately prior to the occurrence of the error, to the detecting device control portion 32. Then, the recovery processing portion 110 returns control to the control system in order to perform an ordinary radiographic image capturing process. As a result, a radiographic image capturing process is performed at the irradiation energy level immediately prior to the occurrence of the error. Thereafter, a radiographic image capturing process is performed at an irradiation energy level (a radiation dose and a frame rate) which is newly set by the operator.

In a case where the error watching portion 104 judges that an error has occurred, the system control portion 14 controls the console 16 to display on the monitor 18 the radiographic image information that was acquired immediately prior to the occurrence of the error. The image information is displayed at the frame rate immediately prior to the occurrence of the error, during a period from the time at which the error was judged to have occurred until the time at which the radiographic image capturing apparatus 12 recovers from the error.

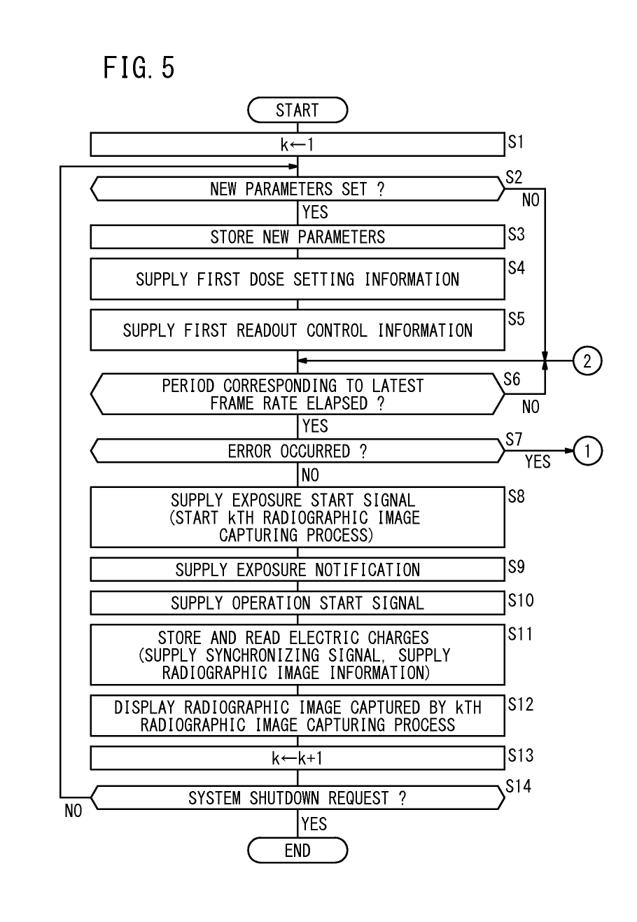

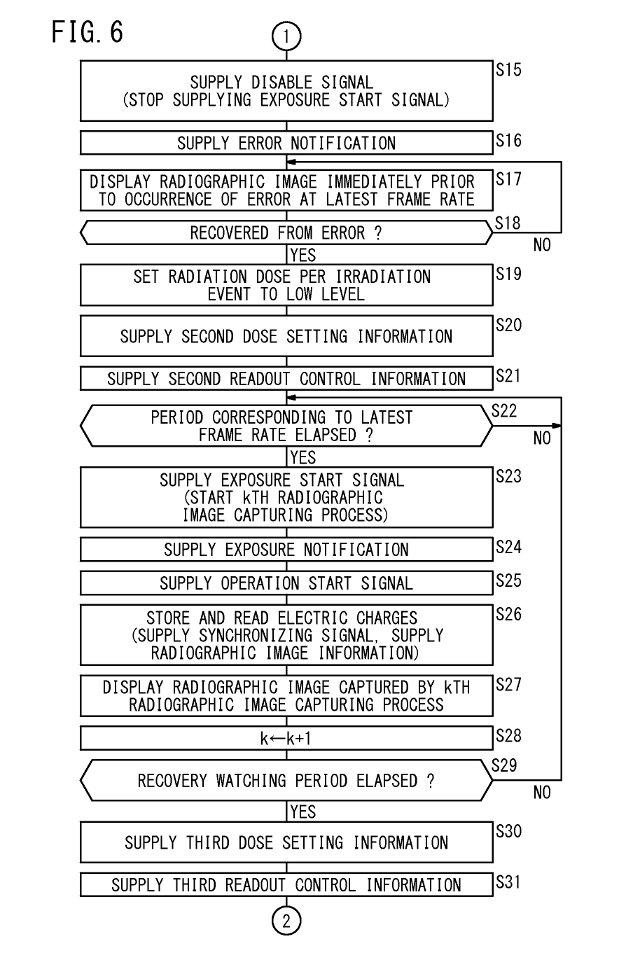

A processing sequence of the first radiographic image capturing system 10A will be described below with reference to the flowcharts shown in FIGS. 5 and 6 and the timing chart shown in FIG. 7.

In step S1 of FIG. 5, the system control portion 14 stores an initial value (=1) in an image capturing counter k.

In step S2, the system control portion 14 judges whether or not parameters (dose of radiation to be applied, frame rate, gain, readout mode, etc.) have been newly set. In a case where the operator has newly set such parameters, then control proceeds to step S3, in which the newly set dose, frame rate, etc., are stored as latest parameters in the parameter history storage portion 102.

In a case where the radiation dose has been newly set, then in step S4, the system control portion 14 supplies first dose setting information Sa1, which includes information (tube voltage, tube current, image capturing time, etc.) concerning the newly set dose, to the radiation device 28. Based on the first dose setting information Sa1 from the system control portion 14, the radiation source control portion 36 of the radiation device 28 sets the radiation dose emitted from the radiation source 34 as a new radiation dose.

In a case where the gain and the readout mode have been newly set, then in step S5, the system control portion 14 supplies first readout control information Sb1, which includes information concerning the newly set gain and the newly set readout mode, through the detecting device control portion 32 to the radiation detecting device 30. Based on the supplied readout control information Sb1, the radiation detecting device 30 sets a gain for the charge amplifiers 66, and sets the type of address signal and the output timing thereof for the address signal generating portion 80.

In step S6, the system control portion 14 judges whether or not the period corresponding to the latest frame rate has elapsed from the start time of the previous radiographic image capturing process. In a case where the value of the counter k is the initial value, or In a case where the period corresponding to the latest frame rate has elapsed from the starting time of the previous radiographic image capturing process, then control proceeds to step S7, in which the error watching portion 104 judges whether or not an error has occurred.

In a case where the error watching portion 104 judges that an error has not occurred, then control proceeds to step S8, in which the system control portion 14 supplies an exposure start signal Sd to the radiation device 28 at the start time of a kth radiographic image capturing process. Based on the exposure start signal Sd supplied from the system control portion 14, the radiation source control portion 36 of the radiation device 28 controls the radiation source 34 to emit radiation 26 at the set radiation dose.

In step S9, the system control portion 14 sends an exposure notification Sf (see FIG. 7) to the detecting device control portion 32, which indicates the start of exposure by the radiation device 28.

In step S10, based on the supplied exposure notification Sf, the detecting device control portion 32 supplies an operation start signal Sg (see FIG. 7) representing the storage of electric charges and the readout of electric charges to the radiation detecting device 30.

In step S11, the radiation detecting device 30 stores electric charges and reads out electric charges based on the operation start signal Sg supplied from the detecting device control portion 32. More specifically, radiation 26 that has passed through the subject 24 initially is converted into visible light by the scintillator. Then, depending on the amount, the visible light is photoelectrically converted into electric charges by the pixels 50, and the electric charges are stored in the pixels 50. At the start of the readout period, the radiation detecting device 30 supplies a synchronizing signal Sh (e.g., a vertical synchronizing signal, see FIG. 7) to the detecting device control portion 32. Based on the supplied synchronizing signal Sh, the detecting device control portion 32 synchronizes the timing at which the radiographic image information is received with the timing at which the radiographic image information is received from the radiation detecting device 30.

During the readout period, the radiation detecting device 30 reads electric charges according to the set readout control information, i.e., information indicating a progressive mode, an interlace mode, or a binning mode, and supplies radiographic image information Da (see FIG. 7) in a FIFO mode, for example, from the memory 82. Radiographic image information Da from the radiation detecting device 30 is supplied through the detecting device control portion 32 to the system control portion 14.

In step S12, the system control portion 14 transfers the supplied radiographic image information Da to the console 16. The console 16 stores the transferred radiographic image information Da in a frame memory, and displays the radiographic image information Da as a radiographic image captured by a kth radiographic image capturing process, i.e., as a radiographic image in a kth frame, on the monitor 18.

In step S13, the value of the counter k is updated by +1.

In step S14, the system control portion 14 judges whether or not there is a system shutdown request. In a case where there is not a system shutdown request, then processing from step S2 is repeated. In this case, insofar as no error has occurred, the operation sequence from step S2 through step S14 is repeated, and the monitor 18 displays a radiographic moving image at the set frame rate.

According to the example shown in FIG. 7, in a case where the radiation dose and the readout mode are changed by a control input from the operator, for example, prior to the starting time to −1 of an (N−1)th (N=2, 3, . . . ) radiographic image capturing process, then the system control portion 14 supplies first dose setting information Sa1, which includes information concerning the newly set radiation dose, to the radiation device 28, and supplies first readout control information Sb1, which includes information concerning the newly set readout mode, through the detecting device control portion 32 to the radiation detecting device 30. In this manner, the radiation device 28 and the radiation detecting device 30 are set to the new radiation dose and the new readout mode.

Thereafter, at the start time tn−1 of the (N−1)th radiographic image capturing process, the system control portion 14 supplies an exposure start signal Sd to the radiation device 28 while sending an exposure notification Sf to the detecting device control portion 32. The system control portion 14 then is supplied with radiographic image information Da that was acquired by the (N−1)th radiographic image capturing process. The system control portion 14 transfers the supplied radiographic image information Da to the console 16, which displays a radiographic image in an (N−1)th frame on the monitor 18. Similarly, at the start time tn of an Nth radiographic image capturing process, after elapse of the latest frame rate Fr from the start time tn−1, the system control portion 14 supplies an exposure start signal Sd to the radiation device 28 while sending an exposure notification Sf to the detecting device control portion 32. The system control portion 14 then is supplied with radiographic image information Da that was acquired by the Nth radiographic image capturing process. The system control portion 14 transfers the supplied radiographic image information Da to the console 16, which displays a radiographic image in an Nth frame on the monitor 18. The above process is repeated to display a radiographic moving image on the monitor 18.

In step S7, in a case where the error watching portion 104 judges that an error has occurred, then control proceeds to step S15 of FIG. 6, during which the radiation emission disabling portion 106 supplies a disable signal Sc for disabling emission of radiation to the radiation device 28. Alternatively, the radiation emission disabling portion 106 may stop supplying the exposure start signal Sd for initiating application of radiation to the radiation device 28. The radiation source control portion 36 of the radiation device 28 stops the radiation source 34 from emitting radiation based on the disable signal Sc from the radiation emission disabling portion 106. In a case where the exposure start signal Sd is not supplied, emission of radiation is disabled.

In step S16, after the disable signal Sc has been supplied from the radiation emission disabling portion 106, or after the exposure start signal Sd has stopped being supplied from the radiation emission disabling portion 106, the error notifying portion 108 sends an error notification Se to the detecting device control portion 32. In response to the error notification Se, the detecting device control portion 32 stops controlling at least the radiation detecting device 30. At this time, all of the pixels may be reset.

In step S17, the system control portion 14 controls the console 16 to display the radiographic image immediately prior to the occurrence of the error at the latest frame rate Fr on the monitor 18.

In step S18, the error watching portion 104 judges whether or not the radiographic image capturing apparatus 12 has recovered from the error. In a case where the error watching portion 104 judges that the radiographic image capturing apparatus 12 has not recovered from the error, control returns to step S17, thus repeating the process of displaying the radiographic image immediately prior to the occurrence of the error on the monitor 18. Accordingly, as shown in FIG. 7, for example, the radiographic image immediately prior to the occurrence of the error is displayed at the latest frame rate Fr on the monitor 18, during a period Ta from the time to at which the error was judged to have occurred until the start time tn+1 of the first radiographic image capturing process after recovery from the error.

In a case where the error watching portion 104 judges that the radiographic image capturing apparatus 12 has recovered from the error, then control proceeds to step S19, during which time the low radiation dose setting portion 112 of the recovery processing portion 110 sets the radiation dose per irradiation event from the radiation source 34 to a predetermined level, which is lower than the radiation dose per irradiation event immediately prior to the occurrence of the error (latest radiation dose).

In step S20, the recovery processing portion 110 supplies second dose setting information Sa2, which includes information (tube voltage, tube current, image capturing time, etc.) concerning the low radiation dose set by the low radiation dose setting portion 112, to the radiation device 28. Based on the second dose setting information Sa2 from the system control portion 14, the radiation source control portion 36 of the radiation device 28 sets the radiation dose emitted from the radiation source 34 to a low radiation dose.

In step S21, the recovery processing portion 110 supplies second readout control information Sb2, which includes information concerning a gain and a readout mode for recovery, through the detecting device control portion 32 to the radiation detecting device 30. Based on the supplied second readout control information Sb2, the radiation detecting device 30 sets the gain for the charge amplifiers 66, and sets the type of address signal and the output timing for the address signal generating portion 80.

The signal processing system tends to become unduly burdened in a case where an ordinary readout process (e.g., a progressive readout process) is carried out after recovery from the error. In view of this drawback, the second readout control information Sb2 includes information for enabling selection of an interlace mode (an odd-numbered row readout mode, an even-numbered row readout mode, an every third row readout mode, etc.), for example. Therefore, any undue burden imposed on the signal processing system of the radiation detecting device 30 is reduced upon recovery from the error. The gain setting information also includes information for enabling setting of the gain of the charge amplifiers 66 to a higher than normal gain.

In step S22, the system control portion 14 judges whether or not the period corresponding to the latest frame rate Fr has elapsed from the start time of the previous radiographic image capturing process. Operations of the radiation device 28 and the radiation detecting device 30 are resumed during a time period corresponding to the latest frame rate Fr, which is elapsing or has elapsed from the start time of the previous radiographic image capturing process.

More specifically, in step S23, the recovery processing portion 110 supplies an exposure start signal Sd to the radiation device 28 at the start time of the kth radiographic image capturing process. Based on the exposure start signal Sd supplied from the system control portion 14, the radiation source control portion 36 of the radiation device 28 controls the radiation source 34 to emit radiation 26 at the previously set low radiation dose.

In step S24, the system control portion 14 sends an exposure notification Sf, which indicates the start of exposure by the radiation device 28, to the detecting device control portion 32.

In step S25, based on the supplied exposure notification Sf, the detecting device control portion 32 supplies an operation start signal Sg, which represents the storage of electric charges and the readout of electric charges, to the radiation detecting device 30.

In step S26, based on the operation start signal Sg supplied from the detecting device control portion 32, the radiation detecting device 30 stores electric charges and reads out electric charges. This operation of the radiation detecting device 30 is the same as the operation carried out in step S11. According to the first embodiment, as described above, since the irradiation energy is set to a low level upon recovery from the error, the radiographic image information, which is read, exhibits a reduced grayscale range. In step S21, for increasing sensitivity, the gain of the charge amplifiers 66 is set to a high level. Consequently, even though the irradiation energy is set to a low level, it is possible to obtain radiographic image information having the same grayscale range as during normal operation thereof.

At the readout period start time, the radiation detecting device 30 supplies a synchronizing signal Sh (e.g., a vertical synchronizing signal) to the detecting device control portion 32. During the readout period, the radiation detecting device 30 reads electric charges according to the set readout control information, i.e., information concerning an interlace mode or the like, and supplies radiographic image information Da in a FIFO mode, for example, from the memory 82. Radiographic image information Da from the radiation detecting device 30 is supplied through the detecting device control portion 32 to the system control portion 14.

In step S27, the system control portion 14 transfers the supplied radiographic image information Da to the console 16. The console 16 stores the transferred radiographic image information Da in the frame memory, and displays the radiographic image information Da as a radiographic image captured by a kth radiographic image capturing process, i.e., as a radiographic image in a kth frame, on the monitor 18.

According to the example shown in FIG. 7, at time tr at which the radiographic image capturing apparatus 12 is judged as having recovered from the error, the system control portion 14 supplies the second dose setting information Sa2, which includes information concerning the low radiation dose, to the radiation device 28. The system control portion 14 also supplies the second readout control information Sb2, which includes the gain setting information and the readout mode information set for recovery, through the detecting device control portion 32 to the radiation detecting device 30. At this time, the radiation device 28 and the radiation detecting device 30 are not set to the low radiation dose, the higher gain, and the readout mode (e.g., an interlace mode).

Thereafter, at the start time tn+1 of an (N+1)th radiographic image capturing process, the system control portion 14 supplies an exposure start signal Sd to the radiation device 28 while also supplying an exposure notification Sf to the detecting device control portion 32. Thereafter, the system control portion 14 is supplied with radiographic image information Da acquired by the (N+1)th radiographic image capturing process (which is carried out at a low irradiation energy). The system control portion 14 transfers the supplied radiographic image information Da to the console 16, which displays the radiographic image information Da as a radiographic image in an (N+1)th frame on the monitor 18. Similarly, at the start time tn+2 of the (N+2)th radiographic image capturing process, after elapse of the latest frame rate Fr from the start time tn+1, the system control portion 14 supplies an exposure start signal Sd to the radiation device 28 while also supplying an exposure notification Sf to the detecting device control portion 32. Thereafter, the system control portion 14 is supplied with radiographic image information Da acquired by the (N+2)th radiographic image capturing process (which is carried out at a low irradiation energy). The system control portion 14 transfers the supplied radiographic image information Da to the console 16, which displays the radiographic image information Da as a radiographic image in an (N+2)th frame on the monitor 18. The above process is repeated to display a radiographic moving image on the monitor 18 after recovery from the error.

In step S28, the value of the counter k is updated by +1.

In step S29, the system control portion 14 judges whether or not a predetermined recovery watching period Tb (see FIG. 7) has elapsed from recovery from the error. In a case where the predetermined recovery watching period Tb has not elapsed, control returns to step S22, and processing from step S22 is repeated.

In a case where the predetermined recovery watching period Tb has elapsed, then control proceeds to step S30, in which the system control portion 14 supplies third dose setting information Sa3, which includes information (tube voltage, tube current, image capturing time, etc.) concerning the radiation dose immediately prior to the occurrence of the error, to the radiation device 28. Based on the third dose setting information Sa3 from the system control portion 14, the radiation source control portion 36 of the radiation device 28 sets the radiation dose emitted from the radiation source 34 to the radiation dose immediately prior to the occurrence of the error.

In step S31, the system control portion 14 supplies third readout control information Sb3, which includes the gain setting information and the readout mode information immediately prior to the occurrence of the error, through the detecting device control portion 32 to the radiation detecting device 30. Based on the supplied third readout control information Sb3, the radiation detecting device 30 sets the gain for the charge amplifiers 66, and the type of address signal and the output timing for the address signal generating portion 80.

Thereafter, control returns to the process from step S6 shown in FIG. 5, and the system control portion 14 controls the radiographic image capturing apparatus 12 to perform an ordinary radiographic image capturing process.

According to the example shown in FIG. 7, at time ta, upon elapse of the recovery watching period Tb from time tr at which the radiographic image capturing apparatus 12 was judged to have recovered from the error, the system control portion 14 supplies the third dose setting information Sa3, which includes information concerning the radiation dose immediately prior to the occurrence of the error, to the radiation device 28. The system control portion 14 also supplies the third readout control information Sb3, which includes the gain setting information and the readout mode information immediately prior to the occurrence of the error, through the detecting device control portion 32 to the radiation detecting device 30. In this manner, the radiation device 28 and the radiation detecting device 30 are set to parameters immediately prior to the occurrence of the error.

Thereafter, at the start time tn+j of an (N+j)th radiographic image capturing process, the system control portion 14 supplies an exposure start signal Sd to the radiation device 28, and also supplies an exposure notification Sf to the detecting device control portion 32. Then, the system control portion 14 is supplied with radiographic image information Da acquired by an (N+j)th radiographic image capturing process. The system control portion 14 transfers the supplied radiographic image information Da to the console 16, which displays the radiographic image information Da as a radiographic image in an (N+j)th frame on the monitor 18. Similarly, at the start time tn+j+1 of an (N+j+1)th radiographic image capturing process, after elapse of the latest frame rate Fr from the start time tn+j, the system control portion 14 supplies an exposure start signal Sd to the radiation device 28, and also supplies an exposure notification Sf to the detecting device control portion 32. Then, the system control portion 14 is supplied with radiographic image information Da acquired by the (N+j+1)th radiographic image capturing process. The system control portion 14 transfers the supplied radiographic image information Da to the console 16, which displays the radiographic image information Da as a radiographic image in an (N+j+1)th frame on the monitor 18. The above process is repeated to display a radiographic moving image on the monitor 18 after recovery from the error.

In a case where the system control portion 14 judges that a system shutdown request has occurred in step S14, the processing sequence of the first radiographic image capturing system 10A is brought to an end.

According to the first radiographic image capturing system 10A, as described above, in a case where an error occurs in the radiographic image capturing apparatus 12, emission of radiation from the radiation source 34 is stopped. However, in a case where the radiographic image capturing apparatus 12 recovers from the error, the radiographic image capturing apparatus 12 can continue carrying out the radiographic image capturing process at a set frame rate in order to capture a radiographic moving image.

Even in a case where the radiographic image capturing apparatus 12 is judged as having recovered from the error, the radiographic image capturing apparatus 12 actually may not have fully recovered from the error, i.e., the error may still remain unremoved. In this case, in a case where the irradiation energy level of the radiation source 34 is set to an ordinary energy level or a high energy level prior to the occurrence of the error while the radiographic image capturing apparatus 12 has not yet fully recovered from the error, then the radiographic image capturing apparatus 12 runs the risk of suffering from a reoccurring error. According to the first radiographic image capturing system 10A, as described above, since the irradiation energy level of the radiation source 34 is set to a preset low energy level, the risk of suffering from a reoccurring error is reduced, and the first radiographic image capturing system 10A can quickly be brought back to a state that enables capturing of a radiographic moving image. In addition, the burden posed on the subject 24 due to undue exposure to radiation 26 is reduced.

According to the first radiographic image capturing system 10A, furthermore, the gain of the charge amplifiers 66 of the radiation detecting device 30 is set to a higher level for increasing sensitivity during the recovery watching period Tb. Consequently, even though the irradiation energy is set to a low level, it is possible to obtain radiographic image information having the same grayscale range as during normal operation thereof. Consequently, a radiographic moving image acquired even at the low irradiation energy level, which is displayed during the recovery watching period Tb, can effectively be used for observation or diagnosis. During the recovery watching period Tb, the readout mode of the radiation detecting device 30 is set to an interlace mode, for example. Therefore, the burden imposed on the signal processing system of the radiation detecting device 30 for reading stored electric charges is reduced, thereby reducing the risk of suffering from a reoccurring error.

At the time that the radiographic image capturing apparatus 12 is judged as having recovered from an error, the system control portion 14 may supply a command to the automatic collimating portion 38 for reducing the area irradiated with radiation 26, so that the area irradiated with radiation 26 can be reduced during the recovery watching period Tb. In this manner, the burden posed on the subject 24 due to undue exposure to radiation 26 is reduced.

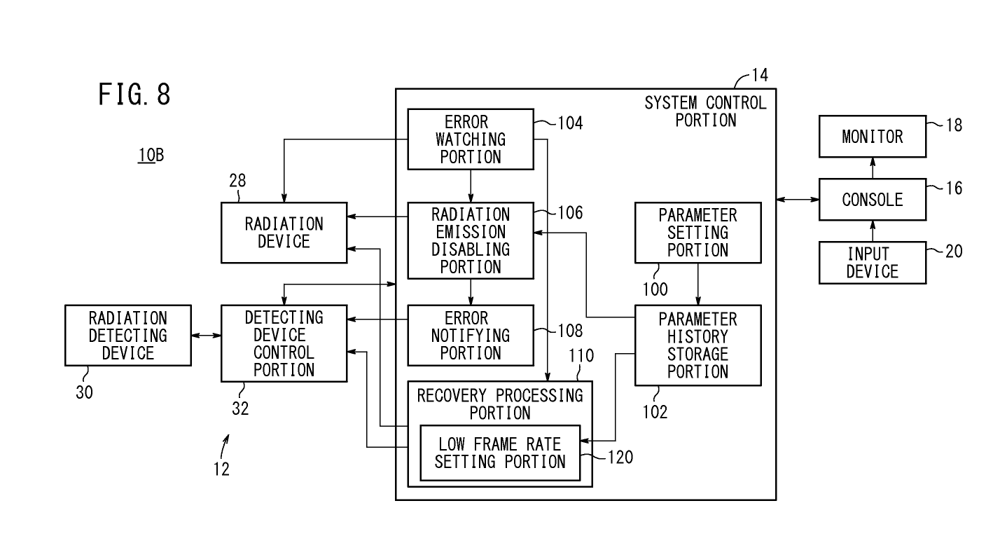

A radiographic image capturing system according to a second embodiment of the present invention (hereinafter referred to as a “second radiographic image capturing system 10B”) will be described below with reference to FIGS. 8 through 10.

The second radiographic image capturing system 10B essentially is of the same configuration as the first radiographic image capturing system 10A, but differs therefrom in that, instead of the low radiation dose setting portion 112, the second radiographic image capturing system 10B has a low frame rate setting portion 120 for setting a low frame rate during the recovery watching period Tb. The low frame rate setting portion 120 sets a frame rate to a level that is in a range from ⅓ to ⅔ of the latest frame rate Fr stored in the parameter history storage portion 102. The low frame rate setting portion 120 may alternatively set a frame rate to a lower ratio, e.g., ⅕ to ⅘. In order to distinguish from the latest frame rate Fr, the frame rate set by the low frame rate setting portion 120 will be referred to as a “low frame rate Fra”.

The processing sequence of the second radiographic image capturing system 10B also differs as to the processes carried out in steps S22 through S29 of FIG. 6, which have been described above.

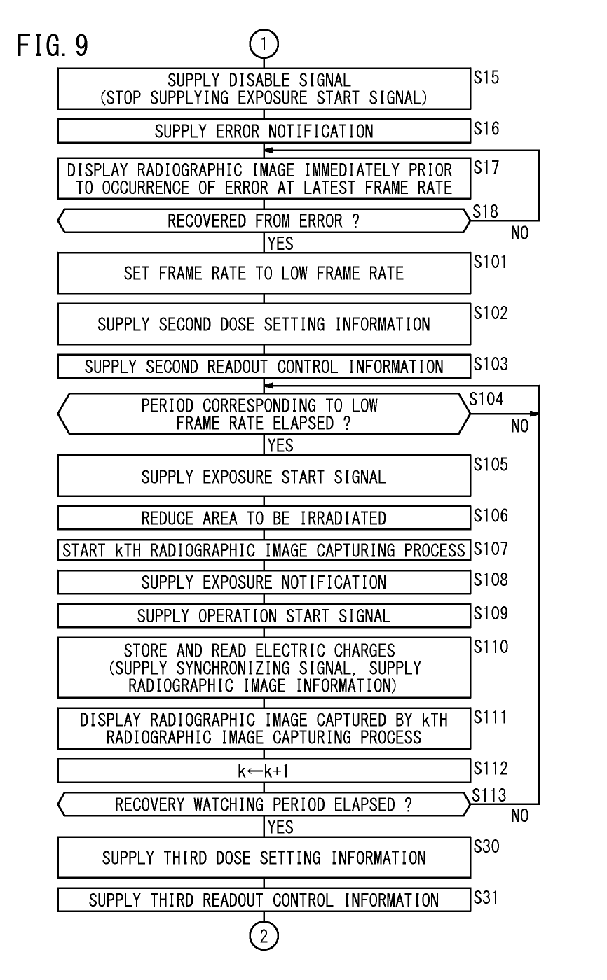

More specifically, in step S101 of FIG. 9, the low frame rate setting portion 120 of the recovery processing portion 110 sets a low frame rate as described above.

In step S102, the recovery processing portion 110 supplies second dose setting information Sa2, which includes information (tube voltage, tube current, image capturing time, etc.) concerning the latest radiation dose stored in the parameter history storage portion 102 and information concerning the low radiation dose that has been set, to the radiation device 28. Based on the second dose setting information Sa2 from the system control portion 14, the radiation source control portion 36 of the radiation device 28 sets a radiation dose, a frame rate, etc.

In step S103, the recovery processing portion 110 supplies second readout control information Sb2, which includes information concerning the gain setting information and the readout mode information upon recovery, through the detecting device control portion 32 to the radiation detecting device 30. Based on the supplied second readout control information Sb2, the radiation detecting device 30 sets the gain for the charge amplifiers 66, and the type of address signal and the output timing for the address signal generating portion 80.

In step S104, the system control portion 14 judges whether or not the period corresponding to the low frame rate Fra has elapsed from the start time of the previous radiographic image capturing process. During a time period corresponding to the low frame rate Fra, which is elapsing or has elapsed from the start time of the previous radiographic image capturing process, control proceeds to the next step S105, in which the recovery processing portion 110 supplies an exposure start signal Sd to the radiation device 28.

In step S106, based on the exposure start signal Sd supplied from the system control portion 14, the radiation source control portion 36 of the radiation device 28 controls the automatic collimating portion 38 in order to reduce the area irradiated with the radiation 26, so as to lie within a range from ¼ to 1/10 of the area irradiated with the radiation 26 immediately prior to the occurrence of the error. The reduction ratio is set in advance by way of simulation or experimentation depending on the body region to be imaged.

In step S107, based on the supplied exposure start signal Sd, the radiation source control portion 36 of the radiation device 28 controls the radiation source 34 to emit radiation 26 at the set radiation dose in a kth radiographic image capturing process.

In step S108, the system control portion 14 sends an exposure notification Sf to the detecting device control portion 32, which indicates the start of exposure by the radiation device 28.

In step S109, based on the supplied exposure notification Sf, the detecting device control portion 32 supplies an operation start signal Sg, which represents the storage of electric charges and the readout of electric charges, to the radiation detecting device 30.

In step S110, the radiation detecting device 30 stores electric charges and reads out electric charges based on the operation start signal Sg supplied from the detecting device control portion 32. This operation of the radiation detecting device 30 is the same as the operation thereof in step S26 of FIG. 6. According to the second embodiment, the gain of the charge amplifiers 66 is not changed, but remains the same as the gain immediately prior to the occurrence of the error.

At the start time of the readout period, the radiation detecting device 30 supplies a synchronizing signal Sh (e.g., a vertical synchronizing signal). In the readout period, the radiation detecting device 30 reads the electric charges according to the instructed readout control information, i.e., an interlace mode or the like, and supplies radiographic image information Da in a FIFO mode, for example, from the memory 82. The radiographic image information Da from the radiation detecting device 30 is supplied through the detecting device control portion 32 to the system control portion 14.

In step S111, the system control portion 14 transfers the supplied radiographic image information Da to the console 16. The console 16 stores the transferred radiographic image information Da in the frame memory, and displays the radiographic image information Da as a radiographic image captured by a kth radiographic image capturing process, i.e., as a radiographic image in a kth frame, on the monitor 18.

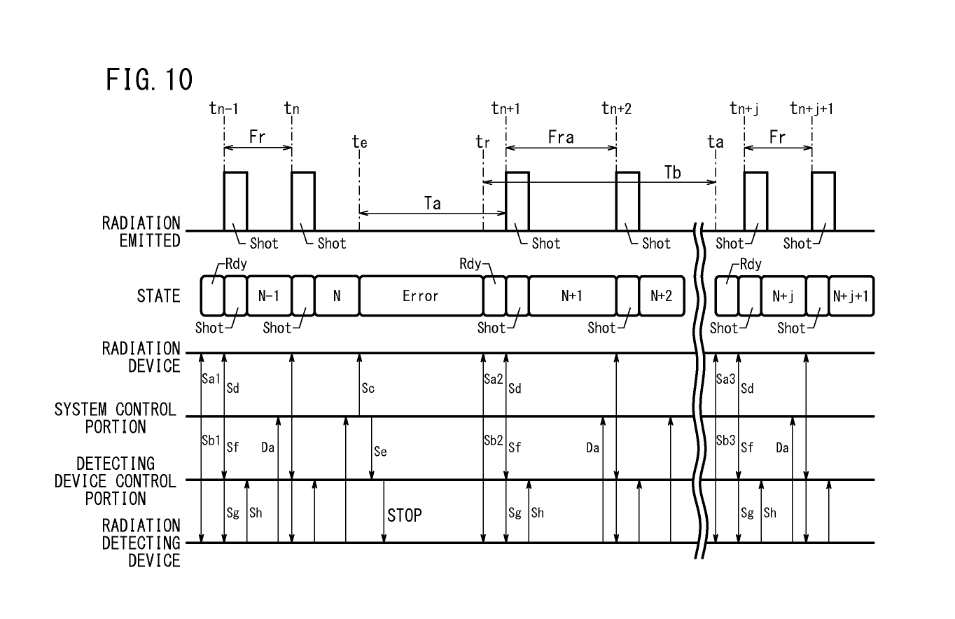

According to the example shown in FIG. 10, at the start time tn+1 of the (N+1)th radiographic image capturing process, for example, after recovery from the error, the system control portion 14 supplies an exposure start signal Sd to the radiation device 28 while also supplying an exposure notification Sf to the detecting device control portion 32. Then, the system control portion 14 is supplied with radiographic image information Da acquired by the (N+1)th radiographic image capturing process (carried out with the latest radiation dose). The system control portion 14 transfers the supplied radiographic image information Da to the console 16, which displays the radiographic image information Da as a radiographic image in an (N+1)th frame on the monitor 18. Upon elapse of a period corresponding to a low frame rate Fra from the start time tn+1 of the (N+1)th radiographic image capturing process, i.e., at the start time tn+2 of the (N+2)th radiographic image capturing process, the system control portion 14 supplies an exposure start signal Sd to the radiation device 28 while also supplying an exposure notification Sf to the detecting device control portion 32. The system control portion 14 is then supplied with radiographic image information Da acquired by the (N+2)th radiographic image capturing process (carried out with the latest radiation dose). The system control portion 14 transfers the supplied radiographic image information Da to the console 16, which displays the radiographic image information Da as a radiographic image in an (N+2)th frame on the monitor 18. The above process is repeated to display a radiographic moving image on the monitor 18 after recovery from the error.

In step S112, the value of the counter k is updated by +1.

In step S113, the system control portion 14 judges whether or not a predetermined recovery watching period Tb has elapsed from recovery from the error. In a case where the predetermined recovery watching period Tb has not elapsed, control returns to step S104, and the process from step S104 is repeated. In a case where the predetermined recovery watching period Tb has elapsed, control proceeds to step S30, in which the system control portion 14 controls the radiographic image capturing apparatus 12 to perform an ordinary radiographic image capturing process. For example, the radiographic image capturing apparatus 12 performs a radiographic image capturing process at the irradiation energy (radiation dose, frame rate) set by the operator, or at the irradiation energy set immediately prior to the occurrence of an error.

With the second radiographic image capturing system 10B, similar to the first radiographic image capturing system 10A, in a case where an error has occurred in at least the radiographic image capturing apparatus 12, the radiation source 34 is controlled to stop emission of radiation. In a case where the radiographic image capturing apparatus 12 has recovered from the error, the radiographic image capturing apparatus 12 continues to perform a radiographic image capturing process at the set low frame rate Fra. In addition, the burden posed on the subject 24 due to undue exposure to radiation 26 is reduced.

In particular, according to the second radiographic image capturing system 10B, upon recovery from an error, the radiation source 34 applies radiation having the latest radiation dose during normal operation. Therefore, the sensitivity of the radiation detecting device 30 is prevented from being lowered, and the radiation detecting device 30 can acquire radiographic image information having the same grayscale range as during normal operation. Consequently, a radiographic moving image, which is displayed during the recovery watching period Tb, can effectively be used for observation or diagnosis.

Furthermore, during the period (recovery watching period Tb) from recovery from the error to restoration of the ordinary radiographic image capturing process, since the area to be irradiated with radiation 26 is reduced, the burden posed on the subject 24 due to undue exposure to radiation 26 is reduced.

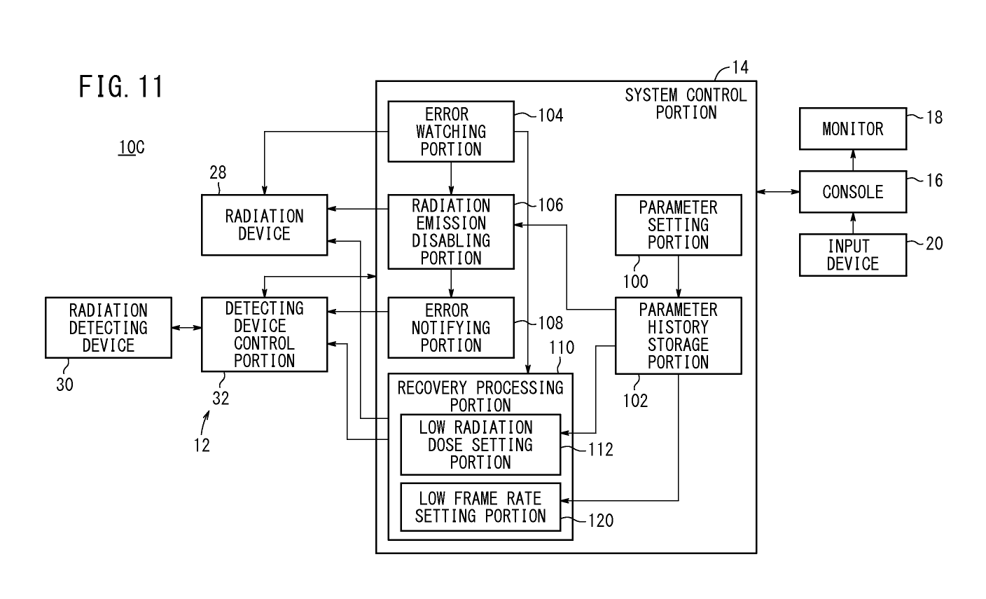

A radiographic image capturing system according to a third embodiment of the present invention (hereinafter referred to as a “third radiographic image capturing system 100”) will be described below with reference to FIG. 11.

The third radiographic image capturing system 100 has a configuration, which combines features from the first radiographic image capturing system 10A and the second radiographic image capturing system 10B.

More specifically, as shown in FIG. 11, a system control portion 14 includes a low radiation dose setting portion 112 and a low frame rate setting portion 120.

The processing sequence of the third radiographic image capturing system′ 100 is similar to the processing sequence of the second radiographic image capturing system 10B, but differs therefrom in the following ways.

The processing sequence of the third radiographic image capturing system 100 differs from the processing sequence of the second radiographic image capturing system 10B, in that in step S19 of FIG. 6, the low radiation dose setting portion 112 sets the dose of a radiation per irradiation event from the radiation source 34 to a level lower than the radiation dose per irradiation event immediately prior to the occurrence of the error (latest radiation dose), and the low frame rate setting portion 120 sets a low frame rate during the recovery watching period Tb. In addition, in step S22, the system control portion 14 judges whether or not the period corresponding to the low frame rate Fra has elapsed from the start time of the previous radiographic image capturing process.

The third radiographic image capturing system 100 offers the same advantages as those of the first radiographic image capturing system 10A and the second radiographic image capturing system 10B.

In particular, since the radiation dose is set to a preset low radiation dose and the frame rate is set to a preset low frame rate Fra for carrying out the radiographic image capturing process upon recovery from the error, the risk of suffering from a reoccurring error is reduced, and the third radiographic image capturing system 100 can quickly be brought back to a state that enables capturing of a radiographic moving image. In addition, the burden posed on the subject 24 due to undue exposure to radiation 26 is reduced. At the time that the radiographic image capturing apparatus 12 is judged as having recovered from an error, the system control portion 14 may supply a command to the automatic collimating portion 38 in order to reduce the area irradiated with radiation 26, so that the area irradiated with radiation 26 can be reduced during the recovery watching period Tb.

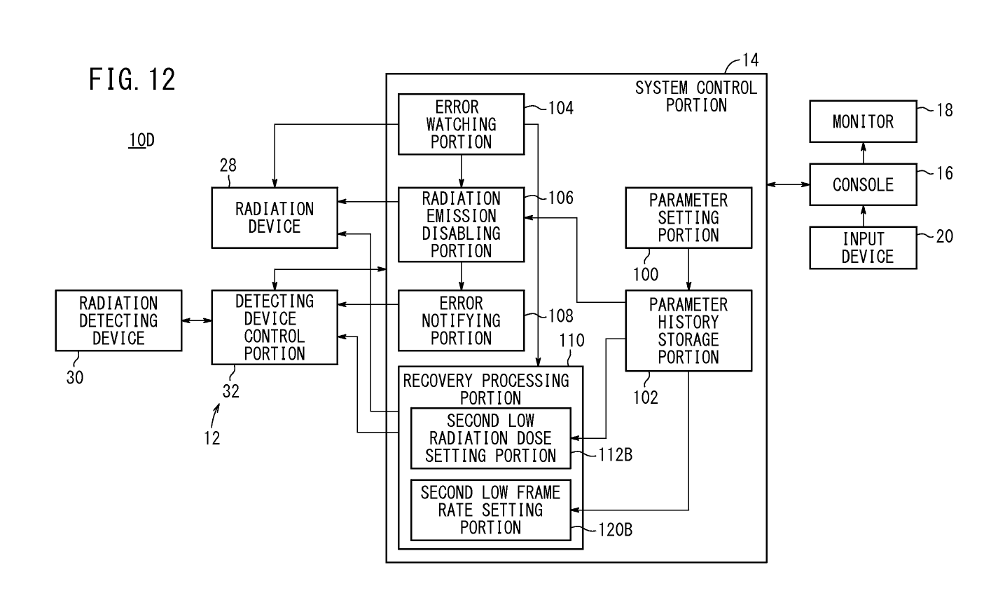

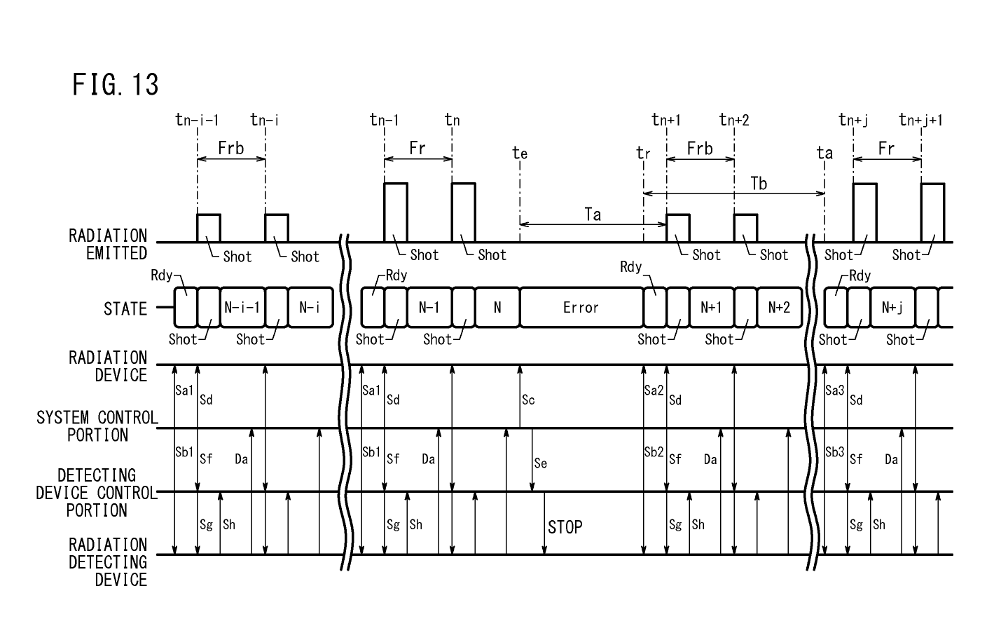

A radiographic image capturing system according to a fourth embodiment of the present invention (hereinafter referred to as a “fourth radiographic image capturing system 10D”) will be described below with reference to FIGS. 12 and 13.

The fourth radiographic image capturing system 10D essentially is of the same configuration as the third radiographic image capturing system 100, but differs therefrom in that the recovery processing portion 110 sets the irradiation energy level to a lowest irradiation energy level from among a plurality of irradiation energy levels set within a predetermined period in the past.

Specifically, the fourth radiographic image capturing system 10D differs in that the fourth radiographic image capturing system 10D has a second low radiation dose setting portion 112B and a second low frame rate setting portion 120B.

The second low radiation dose setting portion 112B reads the lowest radiation dose from among a plurality of radiation doses during a predetermined period in the past, which are stored in the parameter history storage portion 102, and sets the read lowest radiation dose as a low radiation dose during the recovery watching period Tb.

The second low frame rate setting portion 120B reads the lowest frame rate from among a plurality of frame rates during a predetermined period in the past, which are stored in the parameter history storage portion 102, and sets the read lowest frame rate as a low frame rate during the recovery watching period Tb.

The processing sequence of the fourth radiographic image capturing system 10D essentially is the same as the processing sequence of the third radiographic image capturing system 10C described above, and hence redundant descriptions will be omitted. As shown in FIG. 13, in a case where from among the radiographic image capturing processes carried out in a predetermined period in the past, an (N−i−1)th radiographic image capturing process, for example, has a lowest radiation dose and a lowest frame rate Frb, then at the start time tn+1 of an (N+1)th radiographic image capturing process after recovery from the error, the system control portion 14 supplies an exposure start signal Sd to the radiation device 28, and supplies an exposure notification Sf to the detecting device control portion 32. Then, the system control portion 14 is supplied with radiographic image information Da acquired by the (N+1)th radiographic image capturing process (the radiographic image capturing process carried out with the radiation dose in the (N−i−1)th radiographic image capturing process). The system control portion 14 transfers the supplied radiographic image information Da to the console 16, which displays a radiographic image in an (N+1)th frame on the monitor 18. At the time (start time tn+2 of a next (N+2)th radiographic image capturing process) that a period corresponding to the lowest frame rate Frb in the (N−i−1)th radiographic image capturing process has elapsed from the start time of the (N+1)th radiographic image capturing process, the system control portion 14 supplies an exposure start signal Sd to the radiation device 28, and supplies an exposure notification Sf to the detecting device control portion 32. Then, the system control portion 14 is supplied with radiographic image information Da acquired by the (N+2)th radiographic image capturing process (the radiographic image capturing process carried out with the radiation dose in the (N−i−1)th radiographic image capturing process). The system control portion 14 transfers the supplied radiographic image information Da to the console 16, which displays a radiographic image in an (N+2)th frame on the monitor 18. The above process is repeated to display a radiographic moving image on the monitor 18.

Similar to the third radiographic image capturing system 10C, the fourth radiographic image capturing system 10D offers the same advantages as those of the first radiographic image capturing system 10A and the second radiographic image capturing system 10B.

In particular, the radiation dose is set to a lowest radiation dose from among the radiation doses of the radiographic image capturing processes carried out during a predetermined period in the past from the time that an error has occurred. In addition, the frame rate is set to the lowest frame rate Frb from among the frame rates of the radiographic image capturing processes carried out in the predetermined period in the past from the time at which an error has occurred. Thereafter, radiographic image capturing processes are carried out with the lowest radiation dose and the lowest frame rate Frb. Consequently, it is possible to use radiation doses and frame rates, which have proven to be effective. Therefore, the risk of suffering from a reoccurring error is reduced, and the fourth radiographic image capturing system 10D can quickly be brought back to a state for capturing a radiographic moving image. In addition, the burden posed on the subject 24 due to undue exposure to radiation 26 is reduced.

In the fourth radiographic image capturing system 10D, the recovery processing portion 110 includes the second low radiation dose setting portion 112B and the second low frame rate setting portion 120B. However, either one of the second low radiation dose setting portion 112B and the second low frame rate setting portion 120B may be dispensed with.

In a case where the second low frame rate setting portion 120B is dispensed with, and only the second low radiation dose setting portion 112B is used, then similar to the case of the first radiographic image capturing system 10A, the fourth radiographic image capturing system 10D may use the latest frame rate Fr. Alternatively, similar to the case of the second radiographic image capturing system 10B, the fourth radiographic image capturing system 10D may include the low frame rate setting portion 120 and use the low frame rate Fra set by the low frame rate setting portion 120.

Similarly, in a case where the second low radiation dose setting portion 112B is dispensed with, and only the second low frame rate setting portion 120B is used, then similar to the case of the second radiographic image capturing system 10B, the fourth radiographic image capturing system 10D may use the latest radiation dose. Alternatively, similar to the case of the first radiographic image capturing system 10A, the fourth radiographic image capturing system 10D may include the low radiation dose setting portion 112 and use the low radiation dose set by the low radiation dose setting portion 112.

With the first radiographic image capturing system 10A, the second radiographic image capturing system 10B, the third radiographic image capturing system 10C, and the fourth radiographic image capturing system 10D, during the recovery watching period Tb, the dose of radiation 26 from the radiation source 34 per irradiation event is set to a level that is lower than the dose of radiation 26 from the radiation source 34 per irradiation event prior to the occurrence of the error. In addition, the number of irradiation events per unit time performed by the radiation source 34 is set to a value that is lower than the number of irradiation events per unit time prior to the occurrence of the error. Accordingly, radiographic image capturing processes are performed with the radiation dose and the number of irradiation events that have been set in the foregoing manner. Alternatively, during the recovery watching period Tb, the total irradiation energy level per unit of the radiation source 34 may be set to a low level, and radiation may be emitted continuously from the radiation source 34 during the radiographic image capturing process.

The radiographic image capturing systems and the radiographic image capturing methods according to the present invention are not limited to the aforementioned embodiments. Various arrangements may be adopted without departing from the scope of the present invention.

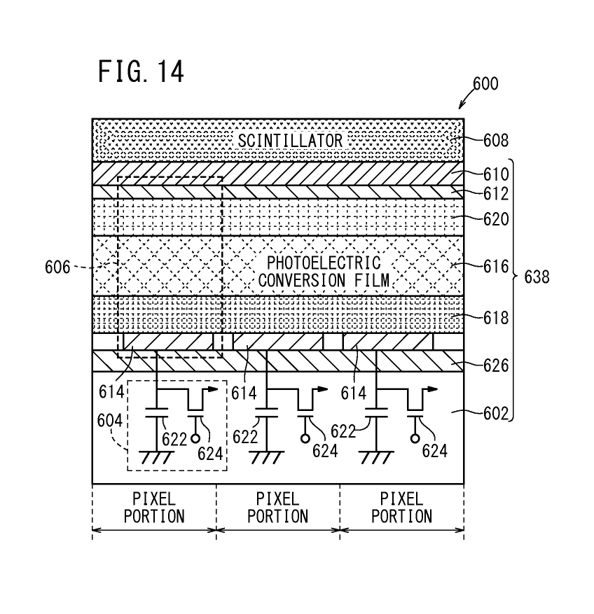

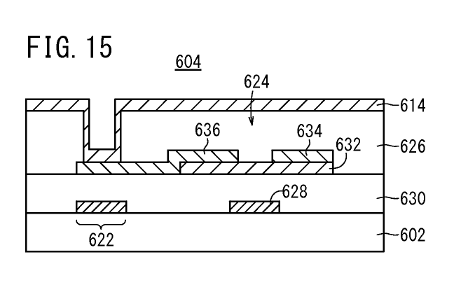

For example, the radiation detector 40 may comprise a radiation detector 600 according to the modification shown in FIGS. 14 and 15. FIG. 14 is a schematic cross-sectional view of three pixel portions of the radiation detector 600 according to such a modification.

As shown in FIG. 14, the radiation detector 600 includes a signal output portion 604, a sensor portion 606 (photoelectric transducer), and a scintillator 608, which are deposited successively on an insulating substrate 602. The signal output portion 604 and the sensor portion 606 jointly make up a pixel portion. The radiation detector 600 includes a matrix of pixel portions arrayed on the insulating substrate 602. In each of the pixel portions, the signal output portion 604 is superposed on the sensor portion 606.

The scintillator 608 is disposed over the sensor portion 606 with a transparent insulating film 610 interposed between the scintillator 608 and the sensor portion 606. The scintillator 608 is in the form of a phosphor film, which emits light converted from radiation 26 that is applied from above (from a side opposite to the substrate 602). Light emitted by the scintillator 608 preferably has a visible wavelength range (from 360 nm to 830 nm). In a case where the radiation detector 600 is used to capture a monochromatic image, then the light emitted by the scintillator 608 preferably includes a green wavelength range.

In a case where X-rays are used as the radiation 26, then the phosphor used in the scintillator 608 preferably includes cesium iodide (CsI), and more preferably, includes CsI(Tl) (thallium-added cesium iodide) which, in a case where irradiated with X-rays, emits light in a wavelength spectrum ranging from 420 nm to 700 nm. Light emitted from CsI(Tl) exhibits a peak wavelength of 565 nm in the visible range.

The scintillator 608 may be formed by depositing CsI(Tl) having a columnar crystalline structure on an evaporation base. In a case where the scintillator 608 is formed by such an evaporation process, then the evaporation base is preferably, but not necessarily, made of Al from the standpoints of X-ray transmittance and reducing cost. In a case where the scintillator 608 is made of GOS, then an evaporation base need not be used, but in this case, the surface of a TFT active matrix substrate may be coated with GOS to form the scintillator 608. Alternatively, a resin base may be coated with GOS to form the scintillator 608, and the scintillator 608 may then be applied to the surface of a TFT active matrix substrate. In this manner, the TFT active matrix substrate can be preserved in the event of a failure of the GOS coating.

The sensor portion 606 includes an upper electrode 612, a lower electrode 614, and a photoelectric conversion film 616, which is disposed between the upper electrode 612 and the lower electrode 614.

Since light emitted by the scintillator 608 must be applied to the photoelectric conversion film 616, the upper electrode 612 preferably is made of an electrically conductive material, which is transparent at least to the wavelength of light emitted by the scintillator 608. More specifically, the upper electrode 612 preferably is made of a transparent conducting oxide (TCO), which exhibits a high transmittance with respect to visible light and has a small resistance value. Although the upper electrode 612 may be made of a thin metal film such as Au or the like, TCO is preferable thereto, because Au tends to have an increased resistance value and exhibits a transmittance of 90% or higher. For example, ITO, IZO, AZO, FTO, SnO₂, TiO₂, ZnO₂, or the like preferably is used as the material of the upper electrode 612. Among these materials, ITO is the most preferable from the standpoints of process simplification, low resistance, and transparence. The upper electrode 612 may be a single electrode, which is shared by all of the pixel portions, or may be a plurality of electrodes, each of which are assigned to respective pixel portions.

The photoelectric conversion film 616, which contains an organic photoconductor (OPC), absorbs light emitted from the scintillator 608, and generates electric charges depending on the absorbed light. A photoelectric conversion film 616 that contains an organic photoconductor (organic photoelectric conversion material), exhibits a sharp absorption spectrum in the range of visible light and does not absorb electromagnetic waves other than light emitted from the scintillator 608. Therefore, any noise produced upon absorption of radiation 26 by the photoelectric conversion film 616 is effectively minimized. The photoelectric conversion film 616 may contain amorphous silicon instead of an organic photoconductor. A photoelectric conversion film 616 that contains amorphous silicon exhibits a wide absorption spectrum for efficiently absorbing light emitted from the scintillator 608.

In order for the organic photoconductor of the photoelectric conversion film 616 to absorb light emitted by the scintillator 608 most efficiently, the absorption peak wavelength thereof should be as close as possible to the light emission peak wavelength of the scintillator 608. Although ideally the absorption peak wavelength of the organic photoconductor and the light emission peak wavelength of the scintillator 608 should be in agreement with each other, it is possible for the light emitted by the scintillator 608 to be absorbed efficiently in a case where the difference between the absorption peak wavelength and the light emission peak wavelength is sufficiently small. More specifically, the difference between the absorption peak wavelength of the organic photoconductor and the light emission peak wavelength of the scintillator 608 with respect to the radiation 26 preferably is 10 nm or smaller, and more preferably, is 5 nm or smaller.

Organic photoconductors that meet the above requirements include quinacridone-based organic compounds and phthalocyanine-based organic compounds. Since quinacridone has an absorption peak wavelength of 560 nm in the visible range, in a case where quinacridone is used as the organic photoelectric conversion material and CsI(Tl) is used as the material of the scintillator 608, the difference between the aforementioned peak wavelengths can be reduced to 5 nm or smaller, thus making it possible to substantially maximize the amount of electric charges generated by the photoelectric conversion film 616.

The sensor portion 606 includes an organic layer formed by superposition or mixture of an electromagnetic wave absorption region, a photoelectric conversion region, an electron transport region, a hole transport region, an electron blocking region, a hole blocking region, a crystallization preventing region, an electrode, and an interlayer contact improving region, etc. The organic layer preferably includes an organic p-type compound (organic p-type semiconductor) or an organic n-type compound (organic n-type semiconductor).

An organic p-type semiconductor is a donor organic semiconductor (compound) mainly typified by a hole-transporting organic compound, and refers to an organic compound that tends to donate electrons. More specifically, in a case where two organic materials are placed in contact with each other, one of the organic materials, which has a lower ionization potential, is referred to as a donor organic compound. Any electron-donating organic compounds can be used as the donor organic compound.

An organic n-type semiconductor is an acceptor organic semiconductor (compound) mainly typified by an electron-transporting organic compound, and refers to an organic compound that tends to accept electrons. More specifically, in a case where two organic materials are placed in contact with each other, one of the organic materials, which has a larger electron affinity, is referred to as an acceptor organic compound. Any electron-accepting organic compounds can be used as the acceptor organic compound.

Materials capable of being used as the organic p-type semiconductor and the organic n-type semiconductor, and arrangements thereof with the photoelectric conversion film 616 are disclosed in detail in Japanese Laid-Open Patent Publication No. 2009-032854, and such features will not be described in detail below. The photoelectric conversion film 616 may contain fullerene or carbon nanotubes.

The thickness of the photoelectric conversion film 616 should be as large as possible for the purpose of absorbing light from the scintillator 608. However, in a case where the thickness of the photoelectric conversion film 616 is greater than a certain value, the intensity of the electric field produced on the photoelectric conversion film 616, which is formed by a bias voltage applied from opposite ends of the photoelectric conversion film 616, becomes reduced and the photoelectric conversion film 616 is unable to collect electric charges. The thickness of the photoelectric conversion film 616 preferably is in a range from 30 nm to 300 nm, more preferably, is in a range from 50 nm to 250 nm, and particularly preferably, is in a range from 80 nm to 200 nm.

The illustrated photoelectric conversion film 616, which is shared by all of the pixel portions, may be divided into a plurality of films assigned to respective pixel portions. The lower electrode 614 comprises a plurality of thin films assigned to respective pixel portions. However, the lower electrode 614 may be a single thin film that is shared by all of the pixel portions. The lower electrode 614 may be made of a transparent or opaque electrically conductive material, preferably aluminum, silver, or the like. The thickness of the lower electrode 614 may be in a range from 30 nm to 300 nm.

In a case where a prescribed bias voltage is applied between the upper electrode 612 and the lower electrode 614, the sensor portion 606 moves one type of electric charges (holes or electrons) that are generated in the photoelectric conversion film 616 toward the upper electrode 612, and moves the other type of electric charges toward the lower electrode 614. With the radiation detector 600 according to the present modification, an interconnection is connected to the upper electrode 612 for applying the bias voltage through the interconnection to the upper electrode 612. The bias voltage has a polarity, which is set to move the electrons generated in the photoelectric conversion film 616 toward the upper electrode 612, and to move the holes toward the lower electrode 614. However, the bias voltage may be of an opposite polarity.

The sensor portion 606 of each pixel portion may include at least the lower electrode 614, the photoelectric conversion film 616, and the upper electrode 612. For preventing dark current from increasing, the sensor portion 606 preferably additionally includes either an electron blocking film 618 or a hole blocking film 620, and more preferably, includes both the electron blocking film 618 and the hole blocking film 620.

The electron blocking film 618 may be disposed between the lower electrode 614 and the photoelectric conversion film 616. In a case where a bias voltage is applied between the lower electrode 614 and the upper electrode 612, the electron blocking film 618 can prevent electrons from being injected from the lower electrode 614 into the photoelectric conversion film 616, thereby preventing dark current from increasing.

The electron blocking film 618 may be made of an electron-donating organic material. The electron blocking film 618 actually is made of a material, which is selected depending on the material of the electrode and the material of the photoelectric conversion film 616 adjacent thereto. A preferable material has an electron affinity (Ea), which is at least 1.3 eV greater than the work function (Wf) of the material of the electrode adjacent thereto, and an ionization potential (Ip), which is equal to or smaller than the Ip of the material of the photoelectric conversion film 616 adjacent thereto. Materials usable as an electron-donating organic material are disclosed in detail in Japanese Laid-Open Patent Publication No. 2009-032854, and such materials will not be described in detail below.

The thickness of the electron blocking film 618 preferably is in a range from 10 nm to 200 nm, more preferably, is in a range from 30 nm to 150 nm, and particularly preferably, is in a range from 50 nm to 100 nm, in order to reliably achieve a dark current reducing capability and to prevent the photoelectric conversion efficiency of the sensor portion 606 from being lowered.

The hole blocking film 620 may be disposed between the photoelectric conversion film 616 and the upper electrode 612. In a case where a bias voltage is applied between the lower electrode 614 and the upper electrode 612, the hole blocking film 620 can prevent holes from being injected from the upper electrode 612 into the photoelectric conversion film 616, thereby preventing dark current from increasing.

The hole blocking film 620 may be made of an electron-accepting organic material. The thickness of the hole blocking film 620 preferably is in a range from 10 nm to 200 nm, more preferably, is in a range from 30 nm to 150 nm, and particularly preferably, is in a range from 50 nm to 100 nm, in order to reliably achieve a dark current reducing capability and to prevent the photoelectric conversion efficiency of the sensor portion 606 from being lowered.

The hole blocking film 620 actually is made of a material, which is selected depending on the material of the electrode and the material of the photoelectric conversion film 616 adjacent thereto. A preferable material has an ionization potential (Ip), which is at least 1.3 eV greater than the work function (Wf) of the material of the electrode adjacent thereto, and an electron affinity (Ea), which is equal to or greater than the Ea of the material of the photoelectric conversion film 616 adjacent thereto. Materials usable as an electron-accepting organic material are disclosed in detail in Japanese Laid-Open Patent Publication No. 2009-032854, and such materials will not be described in detail below.

For setting a bias voltage so as to move holes, from among the electric charges generated in the photoelectric conversion film 616, toward the upper electrode 612, and to move electrons, from among the electric charges generated in the photoelectric conversion film 616, toward the lower electrode 614, the electron blocking film 618 and the hole blocking film 620 may be switched in position. It is not necessary to provide both the electron blocking film 618 and the hole blocking film 620. Either one of the electron blocking film 618 and the hole blocking film 620 may be included in order to provide a certain dark current reducing capability.

As shown in FIG. 15, the signal output portion 604 is disposed on the surface of the substrate 602 in alignment with the lower electrode 614 of each pixel portion. The signal output portion 604 includes a storage capacitor 622 for storing electric charges that have moved to the lower electrode 614, and a TFT 624 for converting electric charges stored in the storage capacitor 622 into electric signals and supplying the electric signals. The storage capacitor 622 and the TFT 624 are disposed in a region that lies underneath the lower electrode 614 as viewed in plan. Such a structure arranges the signal output portion 604 and the sensor portion 606 in a superposed relation in each pixel portion in a thicknesswise direction. In a case where the signal output portion 604 is formed such that the lower electrode 614 fully covers the storage capacitor 622 and the TFT 624, then the planar area of the radiation detector 600 (pixel portions) is minimized.

The storage capacitor 622 is connected electrically to the corresponding lower electrode 614 by an electrically conductive interconnection, which extends through an insulating film 626 that is interposed between the substrate 602 and the lower electrode 614. The interconnection permits electric charges, which are collected by the lower electrode 614, to move to the storage capacitor 622.

The TFT 624 includes a stacked assembly made up of a gate electrode 628, a gate insulating film 630, and an active layer (channel layer) 632. A source electrode 634 and a drain electrode 636 are disposed on the active layer 632 and are spaced from each other with a gap therebetween. The active layer 632 may be made of amorphous silicon, an amorphous oxide, an organic semiconductor material, carbon nanotubes, or the like, for example, although the active layer 632 is not limited to such materials.

The amorphous oxide that constitutes the active layer 632 preferably is an oxide (e.g., In—O oxide) including at least one of In, Ga, and Zn, more preferably, is an oxide (e.g., In—Zn—O oxide, In—Ga—O oxide, or Ga—Zn—O oxide) including at least two of In, Ga, and Zn, and particularly preferably, is an oxide including In, Ga, and Zn. An In—Ga—Zn—O amorphous oxide preferably is an amorphous oxide the crystalline composition of which is represented by InGaO₃ (ZnO)ₘ where m represents a natural number smaller than 6, and particularly preferably, is InGaZnO₄. However, the amorphous oxide that constitutes the active layer 632 is not limited to the aforementioned materials.

The organic semiconductor material that constitutes the active layer 632 may be made of a phthalocyanine compound, pentacene, vanadyl phthalocyanine, or the like, although the organic semiconductor material is not limited to such materials. Details concerning the phthalocyanine compound, for example, are disclosed in detail in Japanese Laid-Open Patent Publication No. 2009-212389, and such features will not be described in detail below.

In a case where the active layer 632 including the TFT 624 is made of an amorphous oxide, an organic semiconductor material, or carbon nanotubes, then since the active layer 632 does not absorb radiation 26 such as X-rays or the like, or only absorbs trace amounts of radiation 26, it is possible to effectively reduce noise produced in the signal output portion 604.

In a case where the active layer 632 is made of carbon nanotubes, then the switching rate of the TFT 624 is increased, and the TFT 624 absorbs light in the visible range at a low rate. However, in a case where the active layer 632 is made of carbon nanotubes, it is necessary to separate and extract highly pure carbon nanotubes by way of centrifugal separation or the like, because the performance of the TFT 624 will be greatly reduced in a case where trace metallic impurities become trapped in the active layer 632.

The amorphous oxide, the organic semiconductor material, the carbon nanotubes, and the organic semiconductor material described above can be deposited as films at low temperatures. Therefore, the substrate 602 is not limited to being a highly heat-resistant substrate such as a semiconductor substrate, a quartz substrate, a glass substrate, or the like, but may be a flexible substrate made of plastic, a substrate of aramid fibers, or a substrate of bionanofibers. More specifically, the substrate 602 may be a flexible substrate of polyester such as polyethylene terephthalate, polybutylene phthalate, polyethylene naphthalate, or the like, polystyrene, polycarbonate, polyethersulfone, polyarylate, polyimide, polycycloolefin, norbornene resin, polychlorotrifluoroethylene, or the like. The flexible substrate enables the radiation detector 600 to be light in weight and hence easy to carry.

By making the photoelectric conversion film 616 from an organic photoconductor and making the TFT 624 from an organic semiconductor material, it is possible to grow the photoelectric conversion film 616 and the TFT 624 at a low temperature on a flexible substrate made of plastic (substrate 602), as well as to make the radiation detector 600 thin and lightweight overall. The radiation detecting device 30, which houses the radiation detector 600 therein, can also be make thin and lightweight for making the radiation detecting device 30 more convenient to use outside of hospitals. Since the base of the photoelectric transducing portion is made of a flexible material, which differs from general glass, the radiation detecting device 30 is highly resistant to damage during times that the radiation detecting device 30 is carried or is placed in use.

The substrate 602 may include an insulating layer for making the substrate 602 electrically insulative, a gas barrier layer for making the substrate 602 impermeable to water and oxygen, and an undercoat layer for making the substrate 602 flat or to improve intimate contact thereof with the electrode.

Aramid fibers for use as the substrate 602 are advantageous in that, since a high-temperature process at 200 degrees Celsius can be applied thereto, aramid fibers allow a transparent electrode material to be set at a high temperature for exhibiting lower resistance. Aramid fibers also allow driver ICs to be automatically mounted thereon by a process including a solder reflow process. Furthermore, inasmuch as aramid fibers have a coefficient of thermal expansion close to that of ITO (Indium Tin Oxide) and glass, a substrate made of aramid fibers is less likely to warp and crack after fabrication. In addition, a substrate made of aramid fibers may be made thinner than a glass substrate or the like. The substrate 602 may be in the form of a stacked assembly, which is constituted from aramid fibers and an ultrathin glass substrate.

Bionanofibers are made by compounding a bundle of cellulose microfibrils (bacteria cellulose) produced by bacteria (acetic acid bacteria, Acetobacter Xylinum) and a transparent resin. The bundle of cellulose microfibrils has a width of 50 nm, which is 1/10 of the wavelength of visible light, is highly strong and highly resilient, and is subject to low thermal expansion. Bionanofibers that contain 60% to 70% of fibers and exhibit a light transmittance of about 90% at a wavelength of 500 nm can be produced by impregnating bacteria cellulose with a transparent resin such as an acrylic resin, an epoxy resin, or the like and setting the transparent resin. Bionanofibers have a low coefficient of thermal expansion ranging from 3 ppm to 7 ppm, which is comparable to silicon crystals, a high strength of 460 MPa that matches the strength of steel, a high resiliency of 30 GPa, and are flexible. Therefore, in a case where the substrate 602 is made of bionanofibers, the substrate 602 can be thinner than glass substrates or the like.

According to the present modification, the signal output portion 604, the sensor portion 606, and the transparent insulating film 610 are formed successively on the substrate 602. Thereafter, the scintillator 608 is bonded above the substrate 602 by an adhesive resin that exhibits low light absorption, thereby completing the radiation detector 600.

With the radiation detector 600 according to the above modification, since the photoelectric conversion film 616 is made of an organic photoconductor and the active layer 632 that includes the TFT 624 is made of an organic semiconductor material, the photoelectric conversion film 616 and the signal output portion 604 absorb almost no radiation 26. Therefore, any reduction in sensitivity to radiation 26 is minimized.

The organic semiconductor material, which includes the active layer 632 made up of the TFT 624, and the organic photoconductor, which includes the photoelectric conversion film 616, can be grown as films at low temperature. Therefore, the substrate 602 can be made from plastic resin, aramid fibers, or bionanofibers, which absorb only a small amount of radiation 26. Thus, any reduction in sensitivity to radiation 26 can be further minimized.

In a case where the radiation detector 600 is placed in the housing and is bonded to the wall that forms the irradiation surface, and in a case where the substrate 602 is made of plastic resin, aramid fibers, or bionanofibers, which are highly rigid, then since the radiation detector 600 exhibits increased rigidity, the wall of the housing that forms the irradiation surface can be made thinner. Further, in a case where the substrate 602 is made of plastic resin, aramid fibers, or bionanofibers, which are highly rigid, then since the radiation detector 600 itself is flexible, the radiation detector 600 is less likely to become damaged as a result of impacts applied to the irradiation surface.

The radiation detector 600 may be arranged in the following ways.

(1) The photoelectric conversion film 616 may be made of an organic photoconductor material, and the TFT layer 638 may be constructed to incorporate CMOS sensors therein. Since only the photoelectric conversion film 616 is made of an organic photoconductor material, the TFT layer 638 including the CMOS sensors may not be flexible.

(2) The photoelectric conversion film 616 may be made of an organic photoconductor material, and the TFT layer 638 may be made flexible by incorporating CMOS circuits having TFTs 624 made of an organic material. The CMOS circuits employ a p-type organic semiconductor material made of pentacene, and an n-type organic semiconductor material made of fluorinated copper phthalocyanine (F₁₆CuPc). In a case where made in this manner, the TFT layer 638 is flexible and can be bent to a smaller radius of curvature, and the TFT layer 638 is effective to significantly reduce the thickness of the gate insulating film, thereby resulting in a lower drive voltage. Furthermore, the gate insulating film, the semiconductor, and the electrodes can be fabricated at room temperature or temperatures that are equal to or lower than 100° C. The CMOS circuits may directly be fabricated on the flexible insulative substrate 602. The TFTs 624, which are made of an organic material, may be microfabricated by a fabrication process according to a scaling law. The substrate 602 may be produced as a flat substrate, which is free of surface irregularities, by coating a thin polyimide substrate with a polyimide precursor, and then heating the applied polyimide precursor to convert the same into polyimide.

(3) The photoelectric conversion film 616 and the TFTs 624, which are made of crystalline Si, may be fabricated on the substrate 602 as a resin substrate by a fluidic self-assembly process. The fluidic self-assembly process allows a plurality of device blocks on the order of microns to be placed at designated positions on the substrate 602. More specifically, the photoelectric conversion film 616 and the TFTs 624, which are constituted as device blocks on the order of microns, are prefabricated on another substrate and then separated from the substrate. Then, the photoelectric conversion film 616 and the TFTs 624 are dipped in a liquid and are spread onto the substrate 602 as a target substrate, so as to be statistically placed in respective positions. The substrate 602 is processed in advance to adapt itself to the device blocks, so that the device blocks can be placed selectively on the substrate 602. Accordingly, the device blocks, i.e., the photoelectric conversion film 616 and the TFTs 624, which are made of an optimum material, can be integrated on an optimum substrate such as a semiconductor substrate, a quartz substrate, a glass substrate, or the like. Therefore, it is possible to integrate optimum device blocks, i.e., the photoelectric conversion film 616 and the TFTs 624, on a non-crystalline substrate such as a flexible substrate made of plastic.

The radiation detector 600 according to the above modification is constructed as a PSS (Penetration Side Sampling) type, i.e., a reverse-side readout type, of radiation detector, in which the sensor portion 606 (the photoelectric conversion film 616), which is positioned remotely from the radiation source 34, converts light emitted from the scintillator 608 into electric charges in order to read a radiographic image. However, the radiation detector 600 is not limited to a PSS type of radiation detector.

A radiation detector may be constructed as an ISS (Irradiation Side Sampling) type, i.e., a face-side readout type, of radiation detector. In such an ISS type of radiation detector, the substrate 602, the signal output portion 604, the sensor portion 606, and the scintillator 608 are stacked in this order along the direction in which radiation 26 is applied. Further, the sensor portion 606, which is positioned close to the radiation source 34, converts light emitted from the scintillator 608 into electric charges in order to read a radiographic image. Since the scintillator 608 usually emits stronger light from the irradiation surface that is irradiated with radiation 26 than from the rear surface thereof, the distance that the light emitted from the scintillator 608 travels until the light reaches the photoelectric conversion film 616 is shorter in a face-side readout type than in a reverse-side readout type of radiation detector. Therefore, the emitted light is scattered and attenuated at a lesser degree, thereby resulting in a radiographic image having higher resolution.

1. A radiographic image capturing system comprising:
a radiographic image capturing apparatus having a radiation device including a radiation source and a radiation detecting device for converting radiation, which is emitted from the radiation source and transmitted through a subject, into radiographic image information; and
a system control portion for controlling the radiographic image capturing apparatus to carry out a radiographic image capturing process at a set frame rate;
wherein the system control portion includes:
a radiation emission disabling portion for stopping the radiation source from emitting radiation in a case where an error has occurred in at least the radiographic image capturing apparatus; and
a recovery processing portion for carrying out a radiographic image capturing process while setting an irradiation energy level of the radiation source to a preset low irradiation energy level upon recovery of the radiographic image capturing apparatus from the error.

2. The radiographic image capturing system according to claim 1, wherein the recovery processing portion sets a radiation dose per irradiation event from the radiation source to a level lower than a radiation dose per irradiation event immediately prior to occurrence of the error.

3. The radiographic image capturing system according to claim 1, wherein the recovery processing portion sets a number of irradiation events per unit time performed by the radiation source to a value lower than a number of irradiation events per unit time prior to occurrence of the error.

4. The radiographic image capturing system according to claim 1, wherein the recovery processing portion sets total irradiation energy level per unit time of the radiation source to a low level.

5. The radiographic image capturing system according to claim 1, wherein the recovery processing portion sets a radiation dose per irradiation event from the radiation source to a level lower than a radiation dose per irradiation event prior to the occurrence of the error, and sets a number of irradiation events per unit time performed by the radiation source to a value lower than a number of irradiation events per unit time prior to the occurrence of the error.

6. The radiographic image capturing system according to claim 1, wherein the recovery processing portion sets the irradiation energy level of the radiation source to a lowest irradiation energy level from among a plurality of irradiation energy levels set within a predetermined period in past.

7. The radiographic image capturing system according to claim 1, wherein the radiographic image capturing apparatus further comprises:
a radiation source control portion for controlling the radiation source based on a command from the system control portion;
wherein the radiation emission disabling portion supplies a disable signal for disabling emission of radiation to the radiation source control portion; and
the radiation source control portion stops the radiation source from emitting radiation based on the disable signal supplied from the radiation emission disabling portion.

8. The radiographic image capturing system according to claim 7, wherein the radiographic image capturing apparatus further comprises:
a detecting device control portion for controlling the radiation detecting device based on a command from the system control portion;
wherein the system control portion sends an error notification to the detecting device control portion after the disable signal has been supplied from the radiation emission disabling portion; and
the detecting device control portion stops controlling at least the radiation detecting device based on the error notification sent from the system control portion.

9. The radiographic image capturing system according to claim 1, wherein the radiographic image capturing apparatus further comprises:
a radiation source control portion for controlling the radiation source based on a command from the system control portion;
wherein the radiation emission disabling portion stops supply of an exposure start signal for emitting radiation to the radiation source control portion.

10. The radiographic image capturing system according to claim 9, wherein the radiographic image capturing apparatus further comprises:
a detecting device control portion for controlling the radiation detecting device based on a command from the system control portion;
wherein the system control portion sends an error notification to the detecting device control portion after the radiation emission disabling portion has stopped supply of the exposure start signal; and
the detecting device control portion stops controlling the radiation detecting device based on the error notification sent from the system control portion.

11. The radiographic image capturing system according to claim 8, wherein based on the recovery from the error, the recovery processing portion supplies information concerning setting of the irradiation energy level of the radiation source to the low irradiation energy level to the radiation device, and supplies parameter information concerning the recovery from the error to the detecting device control portion; and
the system control portion resumes operation of the radiation device and the radiation detecting device.

12. The radiographic image capturing system according to claim 1, further comprising:
a display device for displaying radiographic image information captured by the radiographic image capturing process that is carried out at the set frame rate;
wherein in the case where the error has occurred, the system control portion controls the display device to display radiographic image information captured immediately prior to occurrence of the error at the set frame rate, during a period from the occurrence of the error to the recovery from the error.

13. A radiographic image capturing method for carrying out a radiographic image capturing process at a set frame rate with a radiographic image capturing apparatus including a radiation source and a radiation detecting device for converting radiation, which is emitted from the radiation source and transmitted through a subject, into radiographic image information, comprising the steps of:
stopping the radiation source from emitting radiation in a case where an error has occurred in at least the radiographic image capturing apparatus; and
carrying out a radiographic image capturing process while setting an irradiation energy level of the radiation source to a preset low irradiation energy level upon recovery of the radiographic image capturing apparatus from the error.
